(19) United States
(12) Patent Application Publication
(10) Pub. No.: US 2022/0354970 A1
(43) Pub. Date: Nov. 10, 2022

(54) TRANSGENIC NON-HUMAN ANIMAL CAPABLE OF CONTROLLING EXPRESSION OF TRANSCRIPTION FACTOR RP58

(71) Applicant: TOKYO METROPOLITAN INSTITUTE OF MEDICAL SCIENCE, Tokyo (JP)

(72) Inventors: Haruo OKADO, Tokyo (JP); Hiroko SHIMBO, Tokyo (JP)

(73) Assignee: TOKYO METROPOLITAN INSTITUTE OF MEDICAL SCIENCE, Tokyo (JP)

(21) Appl. No.: 17/628,793

(22) PCT Filed: Jul. 22, 2020
(86) PCT No.: PCT/JP2020/029372

§ 371 Date: Jan. 20, 2022

(30) Foreign Application Priority Data
Jul. 24, 2019 (JP) 2019-136490

(51) Int. Cl. A61K 49/00; A61K 38/17; A61P 25/00; A01K 67/027; A61P 25/28

(52) U.S. Cl. CPC A61K 49/0008; A61K 38/1709; A61P 25/00; A01K 67/0275; A61P 25/28; A01K 2217/203; A01K 2267/0306; A01K 2217/05; A01K 2227/105; A01K 2267/0318

(57) ABSTRACT

Provided is an invention that is based on the novel function of a transcription factor RP58 in cells of the central nervous system. The present invention relates to: a transgenic non-human animal capable of increasing or decreasing the expression of the transcription factor RP58 in cells of the central nervous system of a non-human animal in the nascent stage and/or during and after the developmental stage; and a pharmaceutical composition for use in the treatment or prevention of brain dysfunction, or behavioral disorder, or a disease related thereto, wherein the pharmaceutical composition comprises a transcription factor RP58 protein, or a gene encoding the transcription factor RP58; etc.

### TECHNICAL FIELD

The present invention relates to a transgenic non-human animal capable of controlling (increasing or decreasing) the expression of the transcription factor RP58, etc.

### BACKGROUND ART

From the nascent stage, the transcription factor RP58 (alias: ZNF238, Zfp238, or ZBTB18) is expressed in excitatory neurons, and is important for the proliferation or transition of neurons and projection formation (Non Patent Literature 1: Okado et al., Dev Biol, 2009). RP58 is a responsible gene for 1q44 deletion syndrome that exhibits symptoms such as human mental retardation, agenesis (hypoplasia) of corpus callosum, and epilepsy. It has been reported that a large number of heterozygous mutations of RP58 are found in patients with developmental delays, including intellectual disability (Non Patent Literature 2: Perlman et al., Am J Med Genet A. 2013; Non Patent Literature 3: Westphal et al., Gene, 2017; Non Patent Literature 4: Ehmke et al., Am J Med Genet A. 2017; Non Patent Literature 5: de Munnik et al., Eur J Hum Genet. 2014; Non Patent Literature 6: Rauch et al., Lancet. 2012; Non Patent Literature 7: Srivastava et al., Ann Neurol. 2014; Non Patent Literature 8: Cohen et al., Clin Genet. 2017; Non Patent Literature 9: Deciphering Developmental Disorders Study. Nature. 2017; and Non Patent Literature 10: van der Schoot et al., Mol Genet Genomic Med. 2018). In addition, it has been also reported that the expression level of RP58 is decreased in human aged frontal lobe (Non Patent Literature 11: Lu et al., Nature, 2004). Moreover, it has been suggested that a decrease in the expression level of RP58 promotes acceleration in the malignancy of brain tumor (Non Patent Literature 12: Carro et al., Nature, 2010). These findings suggest that sufficient expression f RP58 is essential for the maintenance of brain functions.

In studies using RP58 animal models, it has been proved using the fetal brain of RP58 knockout mice that a deficiency of RP58 specifically expressed in glutamatergic neurons causes hypoplasia of the cerebral cortex and the hippocampus (Non Patent Literature 1: Okado et al., Dev Biol, 2009). When the molecular mechanism was analyzed by in utero electroporation using a viral vector, it was found that suppression of the transcription of Id1-4 by RP58 is essential f or cell cycle exit occurring upon differentiation of neutrons from progenitor cells, and that RP58 binds to the transcriptional regulatory region of Id1-4 and directly suppresses the transcription thereof (Non Patent Literature 13: Hirai et al., EMBO J, 2012). Moreover, it has been reported that, by using conditional RP58 knockout mice, it was found that suppression of the transcription of Ngn2 and Rnd2 by RP58 is required for the radial migration of neurons after differentiation of the neurons from progenitor cells, and that RP58 directly binds to their promoter and suppresses the transcription (Non Patent Literature 14: Ohtaka-Maruyama et al., Cell Rep, 2013; and Non Patent Literature 15: Heng et al., Cereb Cortex. 2015). Furthermore, microcephaly, agenesis of corpus callosum, and cerebellar hypoplasia were found after the birth of conditional knockout RP58fl/fl:Nestin-Cre mice (Non Patent Literature 16: Xiang C et al., Cell Death Differ. 2012). Further, in conditional knockout RP58fl/fl:Atoh1-CreER mouse models, severe cerebellar hypoplasia was found, and it has been reported that RP58 is required for both glutamatergic and GABAergic neurons (Non Patent Literature 17: Baubet et al., Development, 2012). In these analyses using RP58-deficient or RP58-conditionally-deficient mouse models, the mice from the fetal stage before the weaning stage (i.e., the nascent stage) were used as subjects, and thus, the mice after the weaning stage, namely, the mice in the adolescence, maturity and old age stages (i.e., during and after the developmental stage) have not been subjected to the analyses.

### CITATION LIST

Non Patent Literature 1: Okado H, Ohtaka-Maruyama C, Sugitani Y, Fukuda Y, Ishida R, Hirai S, Miwa A, Takahashi A, Aoki K, Mochida K, Suzuki O, Honda T, Nakajima K, Ogawa M, Terashima T, Matsuda J, Kawano H, Kasai M. The transcriptional repressor RP58 is crucial for cell-division patterning and neuronal survival in the developing cortex. Dev Biol. 2009 Jul. 15; 331(2): 140-51. doi: 10.1016/j.ydbio.2009.04.030.

Non Patent Literature 2: Perlman S J, Kulkarni S, Manwaring L, Shinawi M. Haploinsufficiency of ZNF238 is associated with corpus callosum abnormalities in 1q44 deletions. Am J Med Genet A. 2013 April; 161A(4): 711-6. doi: 10.1002/ajmg.a.35779

Non Patent Literature 3: Westphal D S, Andres S, Beitzel K I, Makowski C, Meitinger T, Hoefele J. Identification of a de novo microdeletion 1q44 in a patient with hypogenesis of the corpus callosum, seizures and microcephaly—A case report. Gene. 2017 Jun. 15;616: 41-44. doi: 10.1016/j.gene.2017.03.025.

Non Patent Literature 4: Ehmke N, Karge S, Buchmann J, Korinth D, Horn D, Reis O, Häßler F. A de novo nonsense mutation in ZBTB18 plus a de novo 15q13.3 microdeletion in a 6-year-old female. Am J Med Genet A. 2017 May; 173(5): 1251-1256. doi: 10.1002/ajmg.a.38145.

Non Patent Literature 5: De Munnik S A, Garcia-Miñaúr S, Hoischen A, van Bon B W, Boycott K M, Schoots J, Hoefsloot L H, Knoers N V, Bongers E M, Brunner H G. A de novo non-sense mutation in ZBTB18 in a patient with features of the 1q43q44 microdeletion syndrome. Eur J Hum Genet. 2014 June; 22(6): 844-6. doi: 10.1038/ejhg.2013.249.

Non Patent Literature 6: Rauch A, Wieczorek D, Graf E, Wieland T, Endele S, Schwarzmayr T, Albrecht B, Bartholdi D, Beygo J, Di Donato N, Dufke A, Cremer K, Hempel M, Horn D, Hoyer J, Joset P, Ropke A, Moog U, Riess A, Thiel C T, Tzschach A, Wiesener A, Wohlleber E, Zweier C, Ekici A B, Zink A M, Rump A, Meisinger C, Grallert H, Sticht H, Schenck A, Engels H, Rappold G, Schröck E, Wieacker P, Riess O, Meitinger T, Reis A, Strom T M. Range of genetic mutations associated with severe non-syndromic sporadic intellectual disability: an exome sequencing study. Lancet. 2012 Nov. 10; 380(9854): 1674-82. doi: 10.1016/S0140-6736(12)61480-9.

Non Patent Literature 7: Srivastava S, Cohen J S, Vernon H, Barañano K, McClellan R, Jamal L, Naidu S, Fatemi A. Clinical whole exome sequencing in child neurology practice. Ann Neurol. 2014 October; 76(4): 473-83. doi: 10.1002/ana.24251.

Non Patent Literature 8: Cohen J S, Srivastava S, Farwell Hagman K D, Shinde D N, Huether R, Darcy D, Wallerstein R, Houge G, Berland S, Monaghan K G, Poretti A, Wilson A L, Chung W K, Fatemi A. Further evidence that de novo missense and truncating variants in ZBTB18 cause intellectual disability with variable features. Clin Genet. 2017 May; 91(5): 697-707. doi: 10.1111/cge.12861.

Non Patent Literature 9: McRae J F et al., Deciphering Developmental Disorders Study. Prevalence and architecture of de novo mutations in developmental disorders. Nature. 2017 Feb. 23; 542(7642): 433-438. doi: 10.1038/nature21062. Epub 2017 Jan. 25.

Non Patent Literature 10: Van der Schoot V, de Munnik S, Venselaar H, Elting M, Mancini G M S, Ravenswaaij-Arts C M A, Anderlid B M, Brunner H G, Stevens S J C. Toward clinical and molecular understanding of pathogenic variants in the ZBTB18 gene. Mol Genet Genomic Med. 2018 May; 6(3): 393-400. doi: 10.1002/mgg3.387. 24.

Non Patent Literature 11: Lu T, Pan Y, Kao S Y, Li C, Kohane I, Chan J, Yankner B A. Gene regulation and DNA damage in the ageing human brain. Nature. 2004 Jun. 24; 429(6994): 883-91.

Non Patent Literature 12: Carro M S, Lim W K, Alvarez M J, Bollo R J, Zhao X, Snyder E Y, Sulman E P, Anne S L, Doetsch F, Colman H, Lasorella A, Aldape K, Califano A, Iavarone A. The transcriptional network for mesenchymal transformation of brain tumours. Nature. 2010 Jan. 21; 463(7279): 318-25. doi: 10.1038/nature08712.

Non Patent Literature 13: Hirai S, Miwa A, Ohtaka-Maruyama C, Kasai M, Okabe S, Hata Y, Okado H. RP58 controls neuron and astrocyte differentiation by downregulating the expression of Id1-4 genes in the developing cortex. EMBO J. 2012 Mar. 7; 31(5): 1190-202. doi: 10.1038/emboj.2011.486.

Non Patent Literature 14: Ohtaka-Maruyama C, Hirai S, Miwa A, Heng J I, Shitara H, Ishii R, Taya C, Kawano H, Kasai M, Nakajima K, Okado H. (2013) RP58 regulates the multipolar-bipolar transition of newborn neurons in the developing cerebral cortex. Cell Rep. 3: 458-71.

Non Patent Literature 15: Heng J I, Qu Z, Ohtaka-Maruyama C, Okado H, Kasai M, Castro D, Guillemot F, Tan S S. The zinc finger transcription factor RP58 negatively regulates Rnd2 for the control of neuronal migration during cerebral cortical development. Cereb Cortex. 2015 March; 25(3): 806-16. doi: 10.1093/cercor/bht277.

Non Patent Literature 16: Xiang C, Baubet V, Pal S, Holderbaum L, Tatard V, Jiang P, Davuluri R V, Dahmane N. RP58/ZNF238 directly modulates proneurogenic gene levels and is required for neuronal differentiation and brain expansion. Cell Death Differ. 2012 April; 19(4): 692-702. doi: 10.1038/cdd.2011.144.

Non Patent Literature 17: Baubet V, Xiang C, Molczan A, Roccograndi L, Melamed S, Dahmane N. Rp58 is essential for the growth and patterning of the cerebellum and for glutamatergic and GABAergic neuron development. Development. 2012 June; 139(11): 1903-9. doi: 10.1242/dev.075606.

Non Patent Literature 18: Vogel-Ciernia A, Wood M A. Examining object location and object recognition memory in mice. Curr Protoc Neurosci. 2014 Oct. 8; 69: 8.31.1-17. doi: 10.1002/0471142301.ns0831s69.

Non Patent Literature 19: Denninger, J. K., Smith, B. M., Kirby, E. D. Novel Object Recognition and Object Location Behavioral Testing in Mice on a Budget. J. Vis. Exp. 2018 (141), e58593, doi:10.3791/58593.

Non Patent Literature 20: Tal Yardeni, Michael Eckhaus, H. Douglas Morris, Marjan Huizing, and Shelley Hoogstraten-Miller. Retro-orbital injections in mice. Lab Anim (NY). 2011 May; 40(5): 155-160. doi:10.1038/laban0511-155.

Non Patent Literature 21: Juan C. Brenes J C, Michael Padilla, M, Jaime Fornaguera, J, A detailed analysis of open-field habituation and behavioral and neurochemical antidepressant-like effects in postweaning enriched rats. Behavioural Brain Research. 2009, 197, 125-137 doi:10.1016/j.bbr.2008.08.014

Non Patent Literature 22: Rojas-Carvajal M, Jaime Fornaguera J, Andrea Mora-Gallegos A, Juan C. Brenes J C Testing experience and environmental enrichment potentiated open-field habituation and grooming behaviour in rats Animal Behavior, 2018, 137, 1-11. doi: org/10.1016/j.anbehay.2018.01.018

Non Patent Literature 23: Saito T, Saido T C. Neuroinflammation in mouse models of Alzheimer's disease. Clin Exp Neuroimmunol. 2018 November; 9(4): 211-218. doi: 10.1111/cen3.12475.

Non Patent Literature 24: Matsuzaki Y, Konno A, Mochizuki R, Shinohara Y, Nitta K, Okada Y, Hirai H. Intravenous administration of the adeno-associated virus-PHP.B capsid fails to upregulate transduction efficiency in the marmoset brain. Neurosci Lett. 2018 Feb. 5; 665: 182-188. doi: 10.1016/j.neulet.2017.11.049.

Non Patent Literature 25: Castillo E, Leon J, Mazzei G, et al. Comparative profiling of cortical gene expression in Alzheimer's disease patients and mouse models demonstrates a link between amyloi-dosis and neuroinflammation. Sci Rep. 2017; 7: 17762. doi: 10.1038/s41598-017-17999-3

### SUMMARY OF INVENTION

Under such circumstances, the present inventor focused on the usefulness of discovering the novel functions of the transcription factor RP58 in cells of the central nervous system such as brain nervous system cells, in particular, the functions of RP58 not only in the nascent stage, but also during and after the developmental stage.

The present invention has been made, while taking into consideration the aforementioned circumstances. The present invention provides, for example, a transgenic non-human animal, a method of screening for a therapeutic or preventive agent against brain dysfunction, etc., and a pharmaceutical composition for use in the treatment or prevention of brain dysfunction, and the like, as described below.

(1) A transgenic non-human animal capable of increasing or decreasing the expression of a transcription factor RP58 in cells of the central nervous system of the non-human animal in the nascent stage and/or during and after the developmental stage.
(2) The transgenic non-human animal according to the above (1), which is capable of increasing or decreasing the expression of the RP58 in cells of the central nervous system of the non-human animal during and after the developmental stage.
(3) The transgenic non-human animal according to the above (1) or (2), which is capable of increasing or decreasing the expression of the RP58, in a desired period in the nascent stage and/or during and after the developmental stage of the non-human animal.
(4) The transgenic non-human animal according to any one of the above (1) to (3), which is capable of increasing or decreasing the expression of the RP58 according to a Tet system.
(5) The transgenic non-human animal according to the above (4), into which a promoter and a tetracycline-controlled transcriptional activator (tTA) gene linked to the promoter so that the tTA gene can function by the promoter, and a Tet operator (tetO) are introduced, and which is capable of increasing the expression of the RP58 by non-administration of doxycycline.
(6) The transgenic non-human animal according to the above (4), into which a promoter and a tetracycline-controlled transcriptional silencer (tTS) gene linked to the promoter so that the tTS gene can function by the promoter, and a Tet operator (tetO) are introduced, and which is capable of decreasing the expression of the RP58 by non-administration of doxycycline.
(7) The transgenic non-human animal according to the above (5) or (6), wherein the promoter is a Camk2a promoter or an Actin promoter.
(8) The transgenic non-human animal according to any one of the above (5) to (7), wherein the expression of the RP58 is maintained at a normal level by administration of doxycycline.

s(9) The transgenic non-human animal according to any one of the above (1) to (8), wherein the cells of the central nervous system are brain nervous system cells.

(10) The transgenic non-human animal according to any one of the above (1) to (9), wherein the non-human animal is a rodent.
(11) The transgenic non-human animal according to the above (10), wherein the rodent is a mouse.
(12) An animal model of brain dysfunction or a disease related thereto, in which the expression of the transcription factor RP58 is decreased in cells of the central nervous system of the transgenic non-human animal according to any one of the above (1) to (4) and (6) to (11).
(13) The animal model according to the above (12), wherein the expression of the RP58 is decreased in cells of the central nervous system of the transgenic non-human animal in during and after the developmental stage.
(14) The animal model according to the above (12) or (13), wherein the brain dysfunction is at least one selected from the group consisting of brain tumor, cognitive dysfunction, developmental disability, intellectual disability, memory impairment, and age-related brain disorder.
(15) A method of screening for a therapeutic or preventive agent against brain dysfunction or a disease related thereto, which comprises a step of decreasing the expression of the transcription factor RP58 in cells of the central nervous system of the transgenic non-human animal according to any one of the above (1) to (4) and (6) to (11), a step of administering a candidate substance to the non-human animal in which the expression of the RP58 is decreased, and a step of evaluating the pathological condition of the brain dysfunction or a disease related thereto in the non-human animal after administration of the candidate substance.
(16) The screening method according to the above (15), wherein the step of decreasing the expression of the RP58 is a step of decreasing the expression of the RP58 in cells of the central nervous system of the transgenic non-human animal in during and after the developmental stage.
(17) The screening method according to the above (15) or (16), wherein the brain dysfunction is at least one selected from the group consisting of brain tumor, cognitive dysfunction, developmental disability, intellectual disability, memory impairment, and age-related brain disorder.
(18) A pharmaceutical composition for use in the treatment or prevention of brain dysfunction or a disease related thereto, wherein the pharmaceutical composition comprises a transcription factor RP58 protein or a nucleic acid encoding the transcription factor RP58.
(19) A pharmaceutical composition for use in the treatment or prevention of brain dysfunction or a disease related thereto, wherein the pharmaceutical composition comprises a therapeutic or preventive agent obtained by the screening method according to any one of the above (15) to (17).
(20) The pharmaceutical composition according to the above (18) or (19), wherein the brain dysfunction is at least one selected from the group consisting of brain tumor, cognitive dysfunction, developmental disability, intellectual disability, memory impairment, and age-related brain disorder.
(21) A method for treating or preventing brain dysfunction or a disease related thereto, which comprises administering the pharmaceutical composition according to any one of the above (18) to (20) to a patient who has or is likely to have brain dysfunction or a disease related thereto.
(22) The therapeutic or preventive method according to the above (21), wherein the brain dysfunction is at least one selected from the group consisting of brain tumor, cognitive dysfunction, developmental disability, intellectual disability, memory impairment, and age-related brain disorder.
(23) An animal model of behavioral disorder or a disease related thereto, in which the expression of the transcription factor RP58 is increased in cells of the central nervous system of the transgenic non-human animal according to any one of the above (1) to (5) and (7) to (11).
(24) The animal model according to the above (23), wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.
(25) A method of screening for a therapeutic or preventive agent against behavioral disorder or a disease related thereto, which comprises a step of increasing the expression of the transcription factor RP58 in cells of the central nervous system of the transgenic non-human animal according to any one of the above (1) to (5) and (7) to (11), a step of administering a candidate substance to the non-human animal in which the expression of the RP58 is increased, and a step of evaluating the pathological condition of the behavioral disorder or a disease related thereto in the non-human animal after administration of the candidate substance.
(26) The screening method according to the above (25), wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.
(27) The screening method according to the above (25) or (26), wherein the candidate substance is a nucleic acid capable of suppressing the function of a gene encoding RP58.
(28) The screening method according to the above (27), wherein the nucleic acid is antisense DNA, siRNA, shRNA, or microRNA.
(29) A pharmaceutical composition for use in the treatment or prevention of behavioral disorder or a disease related thereto, wherein the pharmaceutical composition comprises a therapeutic or preventive agent obtained by the screening method according to any one of the above (25) to (28).
(30) The pharmaceutical composition according to the above (29), wherein the behavioral disorder or a disease related thereto is at least one selected from the group consisting of borderline personality disorder, schizophrenia, depression, bipolar disorder, eating disorder, dissociative disorder, post-traumatic stress syndrome, autism, addiction, and self-injury.
(31) A method for treating or preventing behavioral disorder or a disease related thereto, which comprises administering the pharmaceutical composition according to the above (29) or (30) to a patient who has or is likely to have behavioral disorder or a disease related thereto.
(32) The therapeutic or preventive method according to the above (31), wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.
(33) A method of screening for a substance that improves or reduces cognitive function, which comprises administering a candidate substance to a wild-type non-human animal, or allowing a candidate substance to come into contact with wild-type animal cells, and then screening the substance that improves or reduces cognitive function, using, as an indicator, the expression level of the transcription factor RP58 in the brain of the non-human animal after the administration or in the animal cells after the contact.
(34) An agent for improving or reducing cognitive function, comprising a substance obtained by the screening method according to the above (33).
(35) A method for improving or reducing cognitive function, comprising administering the agent for improving or reducing cognitive function according to the above (34) to a subject.
(36) A method of screening for an environment that improves or reduces cognitive function, which comprises breeding a wild-type non-human animal under a candidate environment, or culturing wild-type animal cells under a candidate environment, and then screening the environment that improves or reduces cognitive function, using, as an indicator, the expression level of the transcription factor RP58 in the brain of the non-human animal after the breeding or in the animal cells after the culture.
(37) A method for improving or reducing cognitive function, comprising providing the environment screened by the method according to the above (36) to a subject.

### ADVANTAGEOUS EFFECTS OF INVENTION

According to the present invention, there can be provided: a transgenic non-human animal capable of increasing or decreasing the expression of a transcription factor RP58 in cells of the central nervous system of a non-human animal in the nascent stage and/or during and after the developmental stage ; an animal model of brain dysfunction or the like, in which the aforementioned non-human animal is used; and further, a method of screening for a therapeutic or preventive agent against brain dysfunction or the like; a pharmaceutical composition for use in the treatment or prevention of the brain dysfunction or the like; etc.

### BRIEF DESCRIPTION OF DRAWINGS

FIG. 1 is a view showing the protocols of an object location recognition test.

FIG. 2 is a view showing the protocols of an object location recognition+novel object recognition test.

FIG. 3 is a schematic view showing an administration method to the orbital sinus of a mouse. The adeno-associated virus vector produced in the Examples of the present application was administered together with Evans blue, using a normal saline as a solvent.

FIG. 4 is a view showing the results of an object location recognition test performed on wild-type male B6 mice with an age of 4 months old (4 mice), 10 months old (3 mice), and 19 months old (4 mice) (±SE, **P<0.05, ***P<0.005, TTEST).

FIG. 5 is a view showing the results obtained by staining the hippocampal and dentate gyms of ActintTS::Rp58 tetO homo or hetero mice (5-month-old), in which the expression of RP58 was decreased after P21, with RP58, NeuN, and DAPI. As control samples, Rp58 tetO homo mice were used.

FIG. 6 is a view showing the results of an object location recognition test performed on RP58 expression decrease models (4- to 5-month-old) after P21 (CTR tetO (n=6, male), ActintTS::RP58tetO Hetero (n=6, male), and ActintTS::RP58tetO Homo (n=8, male), ±SE, **P<0.05, ***P<0.005, TTEST).

FIG. 7 is a view showing the results of an object location recognition test performed on RP58 expression decrease models (4-month-old) after P60 (CTR tetO (male, n=2; female, n=2), ActintTS::RP58tetO Hetero (male, n=2), and ActintTS::RP58tetO Homo (male, n=2; female, n=1)).

FIG. 8 is a view showing the results obtained by staining Camk2tTA:: Rp58 tetO hetero mice (2-month-old), in which the expression of RP58 in mature excitatory neurons RP58 was increased from the early nascent stage, with RP58. As control samples, Rp58 tetO hetero mice were used.

FIG. 9 is a view showing the results of an object location recognition test performed on RP58 expression increase models (4-month-old and 15-month-old) from the early nascent stage.
4-Month-old:
Camk2tTA::RP58tetO Hetero (male, n=3), CTR tetO (male, n=3)
Camk2tTA::RP58tetO Hetero (female, n=7), CTR tetO (female, n=4)
15-Month-old:
Camk2tTA::RP58tetO Hetero (male, n=4), CTR tetO (male, n=5)
Camk2tTA::RP58tetO Hetero (female, n=3), CTR tetO (female, n=5)
±S.E, **P<0.05, ***P<0.005, TTEST

FIG. 10 is a view showing the results of an object location recognition test (OLT, 3rd session) and a novel object recognition test (NORT, 4th session) performed on RP58 expression increase models (10-month-old) from the early nascent stage (Camk2tTA::RP58tetO Hetero (female, n=3), and CTR tetO (female, n=1)).

FIG. 11 is a view showing that RP58 can be overexpressed in the mature neurons of hippocampus and cerebral cortex by intravenous gene transfer with AAV-CaMKIIa-cMyc-RP58. The mature neurons were stained with cMyc, RP58, and NeuN, and as a result, the levels of the former two (cMyc and RP58) were increased. As control samples, mice administered with AAV-CaMKIIa-Gfp were used, and the results obtained by staining with a GFP antibody were shown. GFP can be expressed at a high level in the mature neurons.

FIG. 12 is a view showing the results of an open field test (left view) and the results of 1st session (habituation) of an object location recognition+novel object recognition test (right view), which were performed on mice (CTR+AAV-RP58) prepared by the forced expression of RP58 in 3-month-old normal controls by using adeno-associated virus (AAV-CaMKIIa-cMyc-RP58).
Left view: The horizontal axis indicates a total of 30 minutes from the initial 5 minutes (0-300 sec) to the last 5 minutes (1501-1800 sec), which are indicated with the unit “second.” Habituation is accelerated in mice in which RP58 is forcibly expressed by AAV.
Right view: The OLT & NORT 1^{st} session of the initial 5 minutes (0-300 sec) and the subsequent 5 minutes (301-600 sec). It correspond to the same open field in the second session 1 week later. Habituation is accelerated in mice in which RP58 is forcibly expressed by AAV (orange), as with genetically modified mice in which the expression of RP58 is increased (green).

FIG. 13 shows the results obtained by administering a GFP-expressed or RP58-expressed adeno-associated virus to 3-month-old AD mouse models, and then performing an object location recognition (OLT) and a novel object recognition test (NORT) on the mouse models with 4 months old. Cognitive function, which had been almost lost in the 3-month-old mouse models, was returned to the normal level by overexpression of RP58.

FIG. 14 is a view showing the results obtained by administering an RP58-expressed adeno-associated virus (AAV-CaMKIIa-cMyc-RP58) to 3-month-old Alzheimer's disease mouse models, and then staining the prefrontal cortex of the mouse models one month after the administration (i.e., 4-month-old mice) with the amyloid β marker 82E1 and with the microglia markers Ibal and CD68. As control samples, mice administered with AAV-CaMKIIa-Gfp were used.

FIG. 15 is a view showing the area of amyloid plaque (upper view) and size distribution (lower view) of 3-month-old Alzheimer's disease mouse models 1 month after administration of RP58-expressed adeno-associated virus (AAV-CaMKIIa-cMyc-RP58) (i.e., 4-month-old mice) (FIG. 14). As control samples, mice administered with AAV-CaMKIIa-Gfp were used. In the box plot of the upper view, the upper end, center, and lower end of the box all indicate a quartile, and the x plotted in the box indicates a mean value.

### DESCRIPTION OF EMBODIMENTS

Hereinafter, the present invention will be described in detail. The scope of the present invention is not constrained by these explanations, and the present invention may be modified, as appropriate, and may be carried out within the range that does not impair the spirit of the present invention, even with regard to those other than the following examples. It is to be noted that all publications cited in the present description, such as, for example, prior art documents, and patent documents such as published unexamined patent applications and patent applications, are incorporated herein by reference in their entireties.

### 1. Summary of the Invention

The transcription factor RP58 is mainly expressed in the excitatory neurons of the cerebral cortex. To date, it has been elucidated that the transcription factor RP58 plays a role for generation and migration of neurons in the nascent stage. In order to newly clarify the function of RP58 for cognitive function during and after the developmental stage, the present inventor has produced mice capable of artificially and freely deceasing the expression of RP58, and mice capable of freely increasing (accelerating) the expression of RP58 in the mature excitatory neurons thereof. Regarding the cognitive function tests, an “object location recognition test” was carried out with reference to the method of Vogel-Ciernia and Wood et al. (the above-mentioned Non Patent Literature 18: Vogel-Ciernia and Wood, Curr Protoc Neurosci, 2014), and continuous evaluation of “object location recognition+novel object recognition test s” was carried out with reference to the method of Denninger et al. (the above-mentioned Non Patent Literature 19: Denninge et al., J. Vis. Exp. 2018). As a result, when the expression of RP58 was decreased in mature mice in an amount-dependent manner, and was further approached to age-related cognitive function. On the other hand, when the expression of RP58 was increased (accelerated) in mature excitatory neurons, cognitive function was rather accelerated, and a reduction in cognitive function due to aging was suppressed. From the aforementioned results, it was demonstrated that the transcription factor RP58 controls cognitive function and a change in cognitive function due to aging in an amount-dependent manner.

Moreover, when RP58 was overexpressed in wild-type mice using an adeno-associated virus vector, an acceleration in cognitive function was observed. Furthermore, when RP58 was overexpressed in knock-in mice with a human mutant amyloid precursor protein (APP) (Alzheimer's disease (AD) mouse models) using an adeno-associated virus vector, reduced cognitive function could be recovered.

By the way, the aforementioned transcription factor RP58 has high species preservation, and at a protein level, a variant of 531 amino acids (531 aa) (human: SEQ ID NO: 17, mouse: SEQ ID NO: 29) and a variant of 522 amino acids (522 aa) (human: SEQ ID NO: 19, mouse: SEQ ID NO: 31) are present between humans and mice. Between humans and mice, 99% of amino acids are identified to one another. Besides, the 531 aa variant protein has a structure in which 9 amino acids are added to the N-terminal side of the 522 aa variant protein (i.e., the 5′-terminal side in the corresponding cDNA sequence).

In the case of RP58, Exons 1 to 4 (SEQ ID NOs: 2 to 5, successively from Exon 1) are present in human genomic DNA (here is exemplified the nucleotide sequence (SEQ ID NO: 1) from the 244048939th nucleotide to the 244057476th nucleotide of DNA registered under GenBank Accession Number: NC_000001.11; hereafter, all registration numbers are GenBank Accession Numbers). Also, in mouse genomic DNA (here is exemplified the nucleotide sequence (SEQ ID NO: 6) from the 177442356th nucleotide to the 177450764th nucleotide of DNA registered under NC_000067.6), Exons 1 to 4 (SEQ ID NOs: 7 to 10, successively from Exon 1) are present, and further, Exon 1b (SEQ ID NO: 35) and Exon 2b (SEQ ID NO: 36) are also present. Regarding human RP58, mainly, three mRNA splicing variants (NM_205768.2 (SEQ ID NO: 13), NM_006352.4 (SEQ ID NO: 15), and NM_001278196.1 (SEQ ID NO: 11)) and the like have been reported. Other than these variants, however, several variants have been reported (XM 017000060.1 (SEQ ID NO: 12) and XM_005273006.2 (SEQ ID NO: 14)). On the other hand, regarding mouse RP58, three mRNA splicing variants (NM_001012330.1 (SEQ ID NO: 26), NM 013915.3 (SEQ ID NO: 27), and NM_001355099.1 (SEQ ID NO: 20)), which correspond to the above-described three human variants, have been reported, and other than these variants, several variants have been reported (XM_006496889.3 (SEQ ID NO: 24), XM 006496890.3 (SEQ ID NO: 25), NM_001355100.1 (SEQ ID NO: 22), NM_001355101.1 (SEQ ID NO: 21), and XM_006496896.3 (SEQ ID NO: 23)). Moreover, it is considered that a plurality of unknown or unreported mRNA splicing variants will be present in both humans and mice. In the present invention, with regard to the transcription factor RP58 used in the following various types of explanations, the sequence information of all of the above-described variants, including unknown or unreported variants, can be referred and/or adopted.

### 2. Transgenic Non-Human Animal

As described above, the transgenic non-human animal of the present invention is a transgenic non-human animal, in which the expression of the transcription factor RP58 can be increased or decreased in cells of the central nervous system of the non-human animal in the nascent stage and/or the during and after developmental stage. Among others, the present transgenic non-human animal is preferably a transgenic non-human animal, in which the expression of RP58 can be increased or decreased in cells of the central nervous system of the non-human animal during and after the developmental stage. Moreover, the present transgenic non-human animal is preferably a transgenic non-human animal, in which the expression of RP58 can be increased or decreased in a desired period of time in the above-described nascent stage and/or during and after the above-described developmental stage, namely, an increase or decrease in the expression of RP58 can be artificially and freely adjusted and/or controlled. Herein, in the present invention, the nascent stage means a period of time from the fetal stage to before the weaning stage (less than the weaning stage), whereas during and after the developmental stage means a period of time after the weaning stage, namely, a period of time including the adolescence, maturity and old age stages.

As mentioned above, conventionally, studies regarding the analysis of the function of the transcription factor RP58 have intended to target only the nascent stage. Thus, the analysis of during and after the developmental stage has not been carried out, and the necessity thereof has not been considered. Hence, the transgenic non-human animal capable of artificially and freely adjusting and/or controlling (i.e., capable of increasing or decreasing) the expression of RP58 in a desired period of time not only in the nascent stage but also during and after the developmental stage of the non-human animal is highly useful as a transgenic non-human animal capable of evaluating the function of RP58, which has not previously been known.

Herein, the method and/or means for artificially and freely adjusting and/or controlling (i.e., increasing or decreasing) the expression of RP58 in cells of the central nervous system in a desired period of time are not limited, and for example, various types of known gene recombination and/or gene transfer methods, such as methods using a Tet system, a Cre/loxP system, and an adeno-associated virus vector (AAV vector), can be adopted. Among others, the Tet system is preferable in that it can control the expression level of RP58 reversibly and gradually.

The Tet system is generally a system capable of reversibly regulating the expression of a gene of interest by administration of doxycycline (Dox) as an antibiotic tetracycline derivative. This Tet system utilizes a Tet repressor (TetR) and a Tet operator sequence (tetO sequence) functioning in the E. coli tetracycline resistance operon. The Tet system utilizes the properties of the TetR and the tetO sequence, by which TetR binds to the tetO sequence in the absence of Dox (−Dox), but in the presence of Dox (+Dox), Dox binds to the TetR so that the TetR cannot binds to the tetO sequence. In the present invention, in order to freely adjust and/or control (i.e., increase or decrease) the expression of RP58 in a desired period of time, a tetracycline-controlled transcriptional activator (tTA) or a tetracycline-controlled transcriptional silencer (tTS) is preferably used, instead of the above-described Tet repressor (TetR). The tTA is a fusion protein of the TetR and a herpesvirus-derived VP16 transcriptional activity domain (VP16AD). In the absence of Dox (−Dox), the TetR portion binds to the tetO sequence, so as to activate the expression of a gene located downstream thereof. However, in the presence of Dox (+Dox), the TetR portion binds to Dox and the TetR portion cannot bind to the tetO sequence, so that it does not activate the expression of a gene located downstream thereof (i.e., the normal level of expression is maintained). Meanwhile, the tTS is a fusion protein of the TetR and a KRAB silencing domain (SDKid-1). In the absence of Dox (−Dox), the TetR portion binds to the tetO sequence, so that the gene expression is suppressed. However, in the presence of Dox (+Dox), the TetR portion cannot bind to the tetO sequence, and thus, the gene expression cannot be suppressed (i.e., the normal level of expression is maintained). Herein, the promoter operably fused to the tTA gene or the tTS gene is not limited. For example, a Camk2a promoter, an Actin promoter, or the like is preferable. More preferably, the Camk2a promoter is preferably used as a promoter operably fused to the tTA gene, whereas the Actin promoter is preferably used as a promoter operably fused to the tTS gene. Under the control of the Camk2a promoter, the gene can be expressed in mature excitatory neurons. On the other hand, under the control of the Actin promoter, the gene can be expressed in brain neurons as a whole. When the Tet system is adopted and the state in the presence of Dox (+Dox) (in a state in which Dox is administered to the non-human animal) is converted to the state in the absence of Dox (−Dox) (in a state in which Dox is not administered to the non-human animal) in a desired period of time, the expression of RP58 can be increased from the normal level in the case of adopting a transcriptional activator (tTA). On the other hand, in the case of adopting a transcriptional silencer (tTS), the expression of RP58 can be decreased from the normal level.

Moreover, with regard to the method using a Cre/loxP system or the method using an adeno-associated virus vector (AAV vector), these methods can be appropriately utilized to adjust and/or control the expression of RP58, while taking into consideration known procedures and technical contents.

In the present invention, “cells of the central nervous system” are not particularly limited, and examples of the cells of the central nervous system may include all nerve cells and glial cells (e.g., astrocytes, oligodendrocytes, and microglia) present in brain nervous system cells (for example, individual nerves of cerebrum, diencephalon, midbrain and cerebellum, excitatory neurons (presynaptic cells)), and central nervous systems such as medulla oblongata and spinal nerve.

Furthermore, examples of the non-human animal that can be used in the present invention may include mammals other than humans, such as mice, rats, guinea pigs, rabbits, pigs, weasels, dogs, cats, monkeys, sheep, bovines, and horses. Among others, rodents (Rodentia) such as mice, rats and guinea pigs are preferable, and mice are more preferable.

As mentioned above (or as mentioned in the Examples later), the transgenic non-human animal of the present invention shows an aspect in which when the expression of RP58 is decreased (for example, during and after the developmental stage), the cognitive function is reduced dependently on the expression level thereof, and is further approached to age-related cognitive function. Thus, the transgenic non-human animal of the present invention can be used as an animal model of brain dysfunction or a disease related thereto (or a disorder related thereto).

Herein, the brain dysfunction is not limited, and for example, the brain dysfunction is at least one selected from the group consisting of brain tumor, cognitive dysfunction, intellectual disability, developmental disability, memory impairment, and age-related brain disorder (including age-related memory impairment). Herein, the brain tumor is a refractory disease, the frequency of onset of which is increased with aging, such as glioma or glioblastoma. The cognitive dysfunction is a disorder by which cognitive function is reduced. The intellectual disability is a state in which intellectual ability is reduced to an intelligence quotient of less than 70. The developmental disability is a disorder that becomes apparent in the developmental stage, and it includes autism spectrum disorder, attention deficit hyperactivity disorder, and learning disability (the developmental disability may also include intellectual disability in some cases). The memory impairment is a state in which a disorder exists in memory, and it includes dementia, traumatic memory impairment, and dissociative impairment. The age-related brain disorder is a brain disorder caused by aging, and it includes physiological and pathological cases.

Moreover, the disease (or the disorder) related to the above-described brain dysfunction is not limited, and examples of the disease (or the disorder) related to the brain dysfunction may include 1q44 deletion syndrome, Alzheimer's dementia, non-Alzheimer's dementia, cerebrovascular dementia, cognitive impairment due to extrinsic causes, and mixed dementia thereof. Among others, examples of the related disease exhibiting cognitive dysfunction may include diseases classified into neuropsychiatric diseases, neurodegenerative diseases, mitochondrial diseases, refractory epilepsy, endocrine metabolic disorders, infectious diseases, cerebrovascular disorders, neuroimmune diseases, etc. More specific examples of the neuropsychiatric diseases may include schizophrenia, depression, and bipolar disorder. More specific examples of the neurodegenerative diseases may include Parkinson's disease, spinocerebellar degeneration, and progressive supranuclear palsy. More specific examples of the mitochondrial diseases may include mitochondrial encephalomyopathy, lactic acid acidosis, stroke-like attack syndrome (MELAS), chronic progressive external ocular muscle palsy syndrome (CPEO), myoclonus epilepsy associated with ragged-red fibers (MERRF), Lee encephalopathy, and Kearns-Sayre syndrome (KSS). More specific examples of the refractory epilepsy may include West syndrome, Lennox-Gasteau syndrome, and temporal lobe epilepsy. More specific examples of the endocrine metabolic disorders may include diabetes, hypoglycemia, Wernicke-Korsakov syndrome, Pellagra, vitamin B12 deficiency, hypothyroidism, hypothyroidism, distant symptoms of cancer, Cushing syndrome, urinary toxicosis, viscous edema, parathyroid disease, Wilson's disease, and further, amino acid metabolism abnormality, ammonia metabolism abnormality, glucose metabolism abnormality, organic acid metabolism abnormality, fatty acid metabolism abnormality, lipid metabolism abnormality, metal metabolism abnormality, and nucleic acid metabolism abnormality. More specific examples of the infectious diseases may include bacterial meningitis, fungal meningitis, encephalitis, AIDS encephalopathy, and Kreuzfeld-Jakob disease. More specific examples of the cerebrovascular disorders may include arteriosclerosis, cerebral arteriosclerosis, chronic cerebral circulatory insufficiency, cerebral bleeding, cerebral infarction, cerebral embolism, submucosal bleeding, chronic subdural bleeding, pseudosphere paralysis, aortic arch syndrome, Binswanger's disease, and arteriovenous malformation-obstructive thrombotic arteritis. More specific examples of the neuroimmune diseases may include multiple sclerosis and Bechet syndrome. Other examples of the related disease may include normal pressure hydrocephalus, camp syndrome, systemic erythematosus, fever attack, and cardiac arrest.

Furthermore, when the expression of RP58 is increased in the transgenic non-human animal of the present invention (for example, in the nascent stage or during and after the developmental stage), cognitive function is abnormally accelerated dependently on the expression level thereof, and the transgenic non-human animal shows behavioral disorder such as self-injury. As such, the transgenic non-human animal of the present invention can be used as an animal model of behavioral disorder or a disease related thereto (or a disorder related thereto).

The behavioral disorder in the present invention means abnormal behavior provoked by an abnormality of the central nervous system, and it includes self-injury, hypersensitivity, interpersonal relationship disorder, aggression abnormality, motivation abnormality, ego dysfunction, and stress tolerance disorder. Specific examples of the behavioral disorder or a disease related thereto are not limited. The behavioral disorder or a disease related thereto may be, for example, at least one selected from the group consisting of borderline personality disorder, schizophrenia, depression, bipolar disorder, eating disorder, dissociative disorder, post-traumatic stress syndrome, addiction, autism, and self-injury. Herein, the borderline personality disorder is a disease often found in young women, which is characterized by instable intrapersonal relationship, and this disorder often causes self-injury such as wrist cutting. The schizophrenia is a chronic refractory psychiatric disorder that is mainly developed in persons with an age of 15 to 35 years old, and exhibits cognitive dysfunction, and positive and negative symptoms. The depression is a disease having a periodic depressive condition, and it is a mood disorder that causes delusion of sin, delusion of poverty, and delusion of mind, when the disease becomes severe. The bipolar disorder is a mood disorder that periodically exhibits a manic state and a depressive state, and this disease is developed at a young age and may show grandiose delusions when it is in a manic state. The eating disorder is a refractory psychiatric disorder that is characterized by overeating and vomiting and is often developed in young women, and in particular, adolescent thinness involves significant weight loss. The dissociative disorder is an ego disorder that causes memory impairment and personality change, probably due to great stress. The post-traumatic stress syndrome is a chronic stress disorder in which after a lot of extraordinary stress such as war, disaster, and assault, aftereffects such as depression, sleep disorder, and flashback continue for more than one month. The addiction is a disease in which a patient repeats drug use, gambling, and the like, and feels intolerable without them. The autism is a developmental disability that exhibits commitment, interpersonal relationship disorders, oversensitivity, and the like. The self-injury is an action that is not suicide but intentionally hurts oneself, and is assumed to have functions such as elimination of emptiness, living (to not commit suicide), and seeking help from others. Diseases caused by such self-injury account for 1.3% of the world's disability-adjusted life years (DALY) and is ranked 20th (WHO, 2003). In other words, at present, self-injury itself is understood as a disease.

### 3. Screening Method

As mentioned above, since the transgenic non-human animal of the present invention can be used as an animal model of brain dysfunction or a disease related thereto, the present transgenic non-human animal is useful for the development of a therapeutic agent against the aforementioned dysfunction or a disease related thereto. Hence, the present invention can provide: a method of screening for a therapeutic or preventive agent against brain dysfunction or a disease related thereto, using the above-described animal model; and a therapeutic or preventive agent against brain dysfunction or a disease related thereto, which is obtained by the aforementioned method. Herein, as specific examples of the brain dysfunction or a disease related thereto, the aforementioned explanations can be applied.

In the present invention, the method of screening for a therapeutic or preventive agent against brain dysfunction or a disease related thereto comprises the following steps (a), (b), and (c):

(a) a step of decreasing the expression of the transcription factor RP58 in cells of the central nervous system of the transgenic non-human animal of the present invention (animal model-producing step);

(b) a step of administering a candidate substance to the above-described non-human animal (animal model) in which the expression of RP58 has been decreased (administration step); and

(c) a step of evaluating the pathological condition of brain dysfunction or a disease related thereto in the above-described non-human animal (animal model) after administration of the candidate substance.

The phrase “to evaluate the pathological condition of brain dysfunction or a disease related thereto” means that the phenotype of the pathological condition regarding the brain dysfunction or a disease related thereto in the transgenic non-human animal after administration of the candidate substance is analyzed. This phase means either to conduct a comparative investigation on the pathological condition of the transgenic non-human animal before and after administration of the candidate substance, or to conduct a comparative investigation on the pathological condition between a test animal administered with the candidate substance and a control animal that has not been administered with the candidate substance.

The method of comparing the phenotype of a test animal with the phenotype of a control animal will be described below. The method is not limited, but examples of the method may include an “object location recognition test” and an “object location recognition+novel object recognition test.”

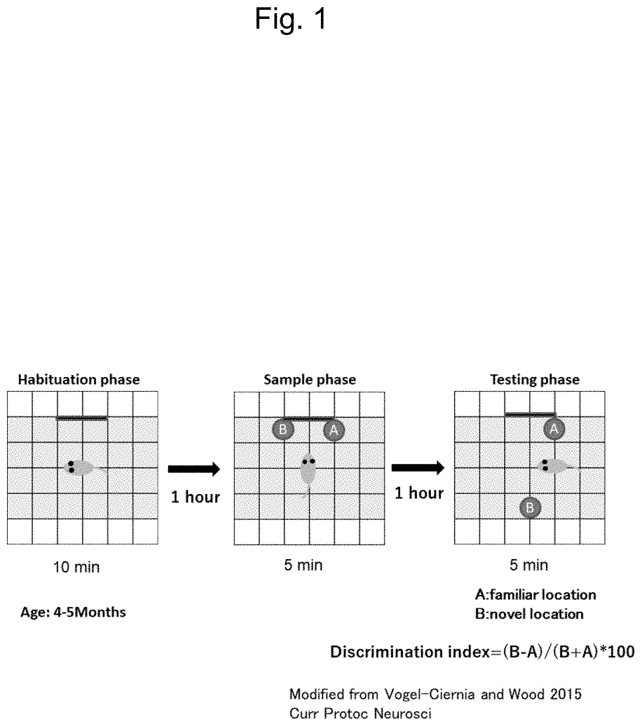

The protocols of the object location recognition test are as shown in FIG. 1. This test is an evaluation method that is based on a principle in which a mouse acts to more often come into contact with an object moved to a new location, if the cognitive function of the mouse is normal. Specifically, when two identical objects are placed in a box, a mouse comes into contact with these objects at almost the same level. The mouse is returned to a home cage, and 1 hour later, one object is moved to another location, and the mouse is then placed in the box again. When the mouse has normal cognitive function, the mouse more often comes into contact with the object moved to another location. When the level of the contact is high, the mouse is evaluated to “have accelerated in terms of cognitive function,” and when the level of the contact is low, the mouse is evaluated to “have reduced in terms of cognitive function.” As the mouse is aged, the mouse rather comes into contact with an object that is not moved. At this time, the mouse is evaluated to have age-related cognitive function.

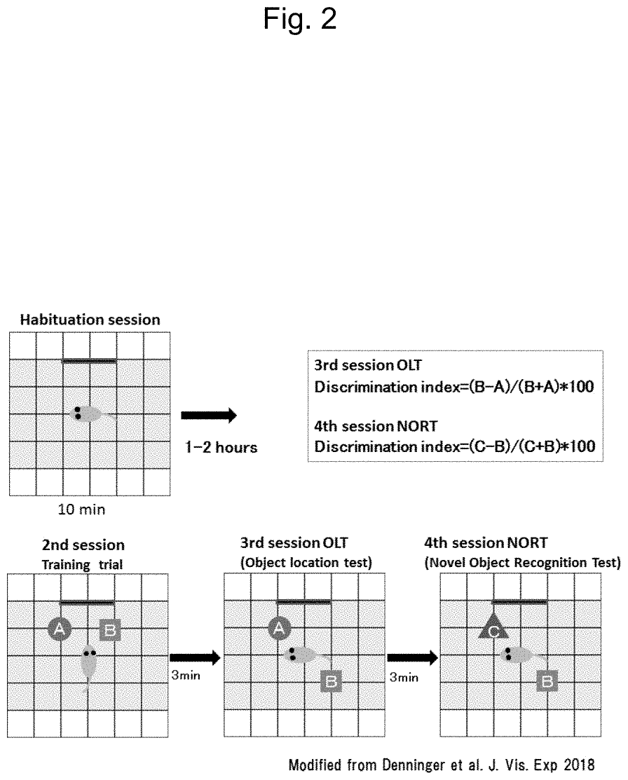

The protocols of the object location recognition+novel object recognition test are as shown in FIG. 2. This test is an evaluation method that is based on a principle in which a mouse acts to more often come into contact with an object moved to a new location, if the cognitive function of the mouse is normal, and acts to more often come into contact with a newly placed object. Specifically, when objects A and B are placed in a box, a mouse comes into contact with these objects at almost the same level. The mouse is returned to a home cage, and 1 hour later, the mouse is placed in the box again in a state in which the object B is moved to another location. When the mouse has normal cognitive function, the mouse more often comes into contact with the object B. When the level of the contact is high, the mouse is evaluated to “have accelerated in terms of cognitive function,” and when the level of the contact is low, the mouse is evaluated to “have reduced in terms of cognitive function.” In addition, the mouse is returned to a home cage, and 1 hour later, the mouse is placed in the box in which an object C is placed instead of the object A. When the mouse has normal cognitive function, the mouse more often comes into contact with the object C. When the level of the contact is high, the mouse is evaluated to “have accelerated in terms of cognitive function,” and when the level of the contact is low, the mouse is evaluated to “have reduced in terms of cognitive function.”

The “control animal” is not limited, as long as it is suitably used in a comparison with a test subject. The control animal may be the transgenic non-human animal of the present invention, in which the expression of RP58 is not decreased (i.e., in which RP58 is expressed at a normal level), or may also be a non-transgenic non-human animal born of the same mother, or may further be a wild-type animal that is not a transgenic animal. Otherwise, a transgenic non-human animal that is not administered with a candidate substance can also be used as a control animal. A wild-type non-human animal born of the same mother as the non-human animal of the present invention, or a wild-type non-human animal of the same species as the non-human animal of the present invention, is preferable as a control animal in that a precise comparative experiment can be carried out using such a control animal.

It is to be noted that the screening method of the present invention may comprise some other steps, as necessary.

Hereafter, the above-described individual steps will be described.

### (a) Animal Model-Producing Step

An animal model of brain dysfunction or a disease related thereto, in which the expression of the transcription factor RP58 is decreased in cells of the central nervous system of the transgenic non-human animal of the present invention, is produced. A period of decrease in the expression of RP58 is not limited, and it may be carried out in the nascent stage, or during and after the developmental stage, or in a period of time that is a combination of the above two stages. It is preferably carried out, for example, during and after the developmental stage. The method of decreasing the expression of RP58 is not limited, and for example, the aforementioned Tet system or the like can be adopted. In this case, continuous administration of doxycycline (Dox) to a transgenic non-human animal can be carried out orally or parenterally, and the administration route is not limited. In both cases, a known administration method, known administration conditions and the like can be adopted. Even regarding an applied dose, the dose can be determined, as appropriate, while taking into consideration the type and condition of a test animal, the type of a candidate substance, etc.

### (b) Administration Step

The candidate substance to be administered to the above-described animal model used as a test animal is not limited. Examples of the candidate substance may include naturally or artificially synthesized, various types of peptides, proteins (including enzymes and antibodies), nucleic acids (polynucleotides (DNA and RNA), oligonucleotides (siRNA, etc.), peptide nucleic acids (PNA), etc.), and low molecular weight, middle molecular weight or high molecular weight organic compounds.

Administration of the candidate substance can be carried out orally or parenterally, and is not limited. In both cases, a known administration method, known administration conditions and the like can be adopted. Even regarding an applied dose, the dose can be determined, as appropriate, while taking into consideration the type and condition of a test animal, the type of a candidate substance, etc.

### (c) Evaluation Step

When a therapeutic or preventive agent against brain dysfunction or a disease related thereto is screened, a test animal that has been administered with a candidate substance is preferably compared and evaluated with a control animal that has not been administered with the candidate substance, in terms of various types of evaluation items (the “object location recognition test” or the “object location recognition+novel object recognition test,” etc.; see FIGS. 1 and 2). When the above-described evaluation items in the test animal can be evaluated to be equivalent to or superior to those in the control animal, the candidate substance can be selected as a therapeutic or preventive agent against brain dysfunction or a disease related thereto.

Moreover, as mentioned above, since the transgenic non-human animal of the present invention can also be used as an animal model of behavioral disorder or a disease related thereto, the present transgenic non-human animal is also useful for the development of a therapeutic agent against the aforementioned behavioral disorder or a disease related thereto. Hence, the present invention can provide: a method of screening for a therapeutic or preventive agent against behavioral disorder or a disease related thereto, using the above-described animal model; and a therapeutic or preventive agent against behavioral disorder or a disease related thereto, which is obtained by the aforementioned method. Herein, as specific examples of the behavioral disorder or a disease related thereto, the aforementioned explanations can be applied.

In the present invention, the method of screening for a therapeutic or preventive agent against behavioral disorder or a disease related thereto comprises the following steps (a), (b), and (c):

(a) a step of increasing (accelerating) the expression of the transcription factor RP58 in cells of the central nervous system of the transgenic non-human animal of the present invention (animal model-producing step);

(b) a step of administering a candidate substance to the above-described non-human animal (animal model) in which the expression of RP58 has been increased (accelerated) (administration step); and

(c) a step of evaluating the pathological condition of behavioral disorder or a disease related thereto in the above-described non-human animal (animal model) after administration of the candidate substance.

The phrase “to evaluate the pathological condition of behavioral disorder or a disease related thereto” means that the phenotype of the pathological condition regarding the behavioral disorder or a disease related thereto in the transgenic non-human animal after administration of the candidate substance is analyzed. This phase means either to conduct a comparative investigation on the pathological condition of the transgenic non-human animal before and after administration of the candidate substance, or to conduct a comparative investigation on the pathological condition between a test animal administered with the candidate substance and a control animal that has not been administered with the candidate substance.

The method of comparing the phenotype of a test animal with the phenotype of a control animal is not particularly limited, and a known evaluation method can be adopted, as appropriate, for each of various types of disorders and/or diseases that are classified into behavioral disorder or a disease related thereto. For instance, in the case of self-injury, a method of evaluating items such as excessive grooming and the resulting alopecia, hair loss and skin damage, tail damage due to biting the tail, severe pulling or biting of the chest skin, and weight loss due to anorexia nervosa.

The “control animal” is not limited, as long as it is suitably used in a comparison with a test subject. The control animal may be the transgenic non-human animal of the present invention, in which the expression of RP58 is not increased (accelerated) (i.e., in which RP58 is expressed at a normal level), or may also be a non-transgenic non-human animal born of the same mother, or may further be a wild-type animal that is not a transgenic animal. Otherwise, a transgenic non-human animal that is not administered with a candidate substance can also be used as a control animal. A wild-type non-human animal born of the same mother as the non-human animal of the present invention, or a wild-type non-human animal of the same species as the non-human animal of the present invention, is preferable as a control animal in that a precise comparative experiment can be carried out using such a control animal.

It is to be noted that the screening method of the present invention may comprise some other steps, as necessary.

Hereafter, the above-described individual steps will be described.

### (a) Animal Model-Producing Step

An animal model of behavioral disorder or a disease related thereto, in which the expression of the transcription factor RP58 is increased (accelerated) in cells of the central nervous system of the transgenic non-human animal of the present invention, is produced. A period of increase (acceleration) in the expression of RP58 is not limited, and it may be carried out, for example, in the nascent stage, or during and after the developmental stage, or in a period of time that is a combination of the above two stages. The method of increasing (accelerating) the expression of RP58 is not limited, and for example, the aforementioned Tet system or the like can be adopted. In this case, continuous administration of doxycycline (Dox) to a transgenic non-human animal can be carried out orally or parenterally, and the administration route is not limited. In both cases, a known administration method, known administration conditions and the like can be adopted. Even regarding an applied dose, the dose can be determined, as appropriate, while taking into consideration the type and condition of a test animal, the type of a candidate substance, etc.

### (b) Administration Step

The candidate substance to be administered to the above-described animal model used as a test animal is not limited. Examples of the candidate substance may include naturally or artificially synthesized, various types of peptides, proteins (including enzymes and antibodies), nucleic acids (polynucleotides (DNA (antisense DNA, etc.) and RNA), oligonucleotides (siRNA, shRNA, microRNA, etc.), peptide nucleic acids (PNA), etc.), and low molecular weight, middle molecular weight or high molecular weight organic compounds.

Administration of the candidate substance can be carried out orally or parenterally, and is not limited. In both cases, a known administration method, known administration conditions and the like can be adopted. Even regarding an applied dose, the dose can be determined, as appropriate, while taking into consideration the type and condition of a test animal, the type of a candidate substance, etc.

### (c) Evaluation Step

When a therapeutic or preventive agent against behavioral disorder or a disease related thereto is screened, a test animal that has been administered with a candidate substance and a control animal that has not been administered with the candidate substance are preferably compared and evaluated, in terms of the conditions and/or pathological conditions of various types of behavioral disorders or diseases related thereto. When the test animal can be evaluated to be equivalent to or superior to the control animal, the candidate substance can be selected as a therapeutic or preventive agent against behavioral disorder or a disease related thereto.

### 4. Pharmaceutical Composition

The present invention can provide a pharmaceutical composition for use in the treatment or prevention of brain dysfunction or a disease related thereto, wherein the pharmaceutical composition comprises a transcription factor RP58 protein. In addition, the present invention can also provide a pharmaceutical composition for use in the treatment or prevention of brain dysfunction or a disease related thereto, and a pharmaceutical composition for use in the treatment or prevention of behavioral disorder or a disease related thereto, wherein each pharmaceutical composition comprises a therapeutic or preventive agent obtained by each of the aforementioned screening methods. As specific examples of the brain dysfunction or a disease related thereto and the behavioral disorder or a disease related thereto, the aforementioned explanations can be applied.

Examples of the RP58 protein that is an active ingredient of the above-described pharmaceutical composition or the substance serving as a therapeutic or preventive agent obtained by each of the aforementioned screening methods are not limited to themselves, and for example, their derivatives can also be used instead of or in combination of them. Examples of the derivatives may include, but are not limited to, those assumed to be derivatives based on common general knowledge to persons skilled in the art, such as those having a chemical structure derived from the original protein, compound or the like.

Examples of the derivatives may include those being subjected to metabolism such as oxidation, reduction, hydrolysis, or conjugation in vivo, and those that are generated as active ingredients by being subjected to metabolism such as oxidation, reduction, or hydrolysis in vivo (so-called prodrugs).

As an active ingredient of the pharmaceutical composition of the present invention, together with the above-described derivatives or prodrugs, or instead of the above-described derivatives or prodrugs, pharmacologically acceptable salts thereof can also be used. Preferred examples of the pharmacologically acceptable salts may include, but are not limited to, hydrohalogenates (e.g., hydrochlorides, hydrobromides, and hydroiodides), inorganic salts (e.g., sulfates, nitrates, perchlorates, phosphates, carbonates, and bicarbonates), organic carboxylates (e.g., acetates, trifluoroacetates, maleates, tartrates, fumarates, and citrates), organic sulfonates (e.g., methane sulfonate, trifluoromethane sulfonate, ethane sulfonate, benzene sulfonate, toluene sulfonate, and camphor sulfonate), amino acid salts (e.g., aspartate and glutamate), quaternary amine salts, alkali metal salts (e.g., sodium salts and potassium salts), and alkaline earth metal salts (e.g., magnesium salts and calcium salts).

Examples of the active ingredient of the pharmaceutical composition of the present invention may also include all isomers possibly generated due to the chemical structure of a compound (for example, geometric isomers, asymmetric carbon-based optical isomers, rotational isomers, stereoisomers, and tautomers), and mixtures of two or more types of these isomers. The active ingredient may be any of an S-form, an R-form, and an RS-form, and is not limited. Furthermore, the active ingredient of the pharmaceutical composition of the present invention may also include those that are present in the form of a hydrate or a solvate, depending on the type thereof. The solvate is not limited, and an example thereof may be a solvate with ethanol.

The amino acid sequence of the RP58 protein that can be used as an active ingredient described above is not limited, and examples thereof may include the amino acid sequences as set forth in SEQ ID NOs: 17, 19, 29, and 31. Examples of the cDNAs encoding these amino acid sequences may include those consisting of the nucleotide sequences as set forth in SEQ ID NOs: 16, 18, 28, and 30.

In the pharmaceutical composition of the present invention, instead of the RP58 protein, or together with the RP58 protein, a protein consisting of an amino acid sequence having an identity of 80% or more to the amino acid sequence of the RP58 protein and having the activity of a transcription factor (i.e., a mutant protein of the RP58 protein) can also be used. Herein, the activity of a transcription factor can be assayed by a method of evaluating transcriptional function, which is called luciferase assay, or an in vivo function evaluation method involving in utero electroporation. This mutant protein may be more preferably a protein consisting of an amino acid sequence having an identity of 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, to the amino acid sequence of the RP58 protein, and having the activity of a transcription factor.

In addition, the mutant protein may also be, for example, a protein having an amino acid sequence comprising a deletion, substitution or addition of one or several amino acids with respect to the amino acid sequence of the RP58 protein, and having the activity of a transcription factor. Herein, the “amino acid sequence comprising a deletion, substitution or addition of one or several amino acids” is not particularly limited. For example, it is an amino acid sequence comprising a deletion, substitution or addition of 1 to 15, 1 to 14, 1 to 13, 1 to 12, 1 to 11, 1 to 10, 1 to 9, 1 to 8, 1 to 7, 1 to 6 (1 to several), 1 to 5, 1 to 4, 1 to 3, 1 or 2, or 1 amino acid, but is not limited thereto. In general, the number of amino acids deleted, substituted or added is preferably as small as possible. Introduction of the mutation such as a deletion, substitution or addition can be carried out using a mutation introduction kit that utilizes site-directed mutagenesis, such as, for example, GeneTailor™ Site-Directed Mutagenesis System (Invitrogen) or TaKaRa Site-Directed Mutagenesis System (Prime STAR (registered trademark) Mutagenesis Basal kit, Mutan (registered trademark)-Super Express Km, etc. (manufactured by Takara Bio Inc.). Moreover, whether or not the concerned peptide is a peptide, into which the above-described mutation such as a deletion, substitution or addition has been introduced, can be confirmed by various types of amino acid sequence determination methods, structural analysis methods using X-ray and NMR, and the like.

The content percentage of the above-described active ingredient, such as an RP58 protein, in the pharmaceutical composition of the present invention is not limited, and it can be determined, as appropriate. The content percentage of the active ingredient can be set within the range of, for example, 0.01% to 99% by weight, 0.01% to 30% by weight, 0.05% to 20% by weight, or 0.1% to 10% by weight, with respect to the total weight of the pharmaceutical composition. By setting the content percentage of the active ingredient within the above-described range, the pharmaceutical composition of the present invention can exhibit therapeutic or preventive effects against brain dysfunction or a disease related thereto, or therapeutic or preventive effects against behavioral disorder or a disease related thereto.

The pharmaceutical composition of the present invention can be administered to a human or a non-human mammal (e.g., a mouse, a rat, a guinea pig, a weasel, a rabbit, sheep, a pig, a bovine, a cat, a dog, a monkey, etc.) used as a test subject, via various administration routes, specifically, via oral administration or parenteral administration (e.g., intravenous injection (IV), intramuscular injection, intraperitoneal injection, subcutaneous injection, rectal administration, and transdermal administration). The present pharmaceutical composition can be formulated into a suitable dosage form using a pharmaceutically acceptable carrier according to a commonly used method, depending on the administration route.

Examples of the dosage form as an oral agent may include tablets, powders, fine granules, granules, coated tablets, capsules, liquids for internal use, suspensions, emulsions, syrups, and troches. Examples of the dosage form as a parenteral agent may include injections (including infusions), inhalants, ointments, nasal drops, and liposomal agents. Besides, when the therapeutic agent and pharmaceutical composition of the present invention are used as various types of the aforementioned oral agents, the therapeutic agent and the pharmaceutical composition can also be used as supplements (corresponding to, for example, functional foods) in some cases.

Examples of the carrier that can be used in formulation of these preparations may include commonly used excipients, binders, disintegrants, lubricants, colorants, and flavoring agents, and also, as necessary, stabilizers, emulsifiers, absorption enhancers, surfactants, pH adjusters, antiseptics, antioxidants, thickeners, wetting agents, surface activators, dispersants, buffers, preservatives, solubilizers, and soothing agents. Known ingredients that can be used as raw materials for pharmaceutical preparations are mixed with the active ingredient, so that a preparation can be formulated according to an ordinary method.

Examples of nontoxic substances that can be used as the aforementioned ingredients may include: animal and vegetable oils, such as soybean oil, beef fat, and synthetic glyceride; hydrocarbons, such as liquid paraffin, squalane, and solid paraffin;

ester oils, such as octyldodecyl myristate and isopropyl myristate; higher alcohols, such as cetostearyl alcohol and behenyl alcohol; silicon resins; silicon oils; surfactants, such as polyoxyethylene fatty acid ester, sorbitan fatty acid ester, glycerin fatty acid ester, polyoxyethylene sorbitan fatty acid ester, polyoxyethylene hydrogenated castor oil, and a polyoxyethylene-polyoxypropylene block copolymer; water-soluble polymers, such as hydroxyethyl cellulose, a polyacrylic acid, carboxyvinyl polymer, polyethylene glycol, polyvinylpyrrolidone, and methyl cellulose; lower alcohols, such as ethanol and isopropanol; polyhydric alcohols (polyols), such as glycerin, propylene glycol, dipropylene glycol, sorbitol, and polyethylene glycol; sugars, such as glucose and sucrose; inorganic powders, such as silicic anhydride, aluminum magnesium silicate, and aluminum silicate; inorganic salts, such as sodium chloride and sodium phosphate; and purified water. All of these substances may also be the salts thereof or the hydrates thereof.

Preferred examples of the excipients may include lactose, fructose, corn starch, white sugar, glucose, mannitol, sorbit, crystalline cellulose, and silicon dioxide. Preferred examples of the binders may include polyvinyl alcohol, polyvinyl ether, methyl cellulose, ethyl cellulose, gum Arabic, tragacanth, gelatin, shellac, hydroxypropylmethyl cellulose, hydroxypropyl cellulose, polyvinylpyrrolidone, a polypropylene glycol-polyoxyethylene block polymer, and meglumine. Preferred examples of the disintegrants may include starch, agar, gelatin powder, crystalline cellulose, calcium carbonate, sodium hydrogen carbonate, calcium citrate, dextrin, pectin, and carboxymethyl cellulose calcium. Preferred examples of the lubricants may include magnesium stearate, talc, polyethylene glycol, silica and hydrogenated vegetable oil. Preferred examples of the colorants may include colorants that have been approved to be added to pharmaceutical products. Preferred examples of the flavoring agents may include cocoa powder, Mentha brain, fragrance powder, mint oil, dragon brain, and cinnamon powder. All of these substances may also be the salts thereof or the hydrates thereof.

Furthermore, the pH adjuster is used in a case where the present pharmaceutical composition is formulated into an injection. The pH adjuster is not particularly limited, as long as it is, for example, a medically, pharmacologically (pharmaceutically) or physiologically acceptable pH adjuster. Specific examples of such a pH adjuster may include: alkali metal hydroxides, such as sodium carbonate, potassium carbonate, sodium ethoxide, potassium butoxide, sodium hydroxide, and potassium hydroxide; alkali metal hydrides, such as sodium hydride and potassium hydride; carbonates of alkali metals or alkaline earth metals; and alkali metal alkoxides. The pH adjuster to be mixed may be used alone or in arbitrary combination of two or more types. When the present pharmaceutical composition is formulated into an injection or the like, it can be appropriately adjusted to an appropriate pH value, using the pH adjuster. In the present invention, the pH value is preferably pH 3 to 6, more preferably pH 3 to 5, further preferably pH 3 to 4.5, and particularly preferably pH 3.5 to 4.5.

Besides, when the present pharmaceutical composition is formulated into an injection, the injection may contain cyclodextrins. Such cyclodextrins are not particularly limited, as long as they are medically, pharmacologically (pharmaceutically) or physiologically acceptable. Examples of such cyclodextrins may include unmodified cyclodextrin and modified cyclodextrin. Examples of the unmodified cyclodextrin may include α-cyclodextrin, β-cyclodextrin, and γ-cyclodextrin. Examples of the modified cyclodextrin may include alkylated cyclodextrin (for example, dimethyl-α-cyclodextrin, dimethyl-β-cyclodextrin, dimethyl-γ-cyclodextrin, etc.), hydroxyalkylated cyclodextrin (for example, hydroxypropyl-α-cyclodextrin, hydroxypropyl-β-cyclodextrin, hydroxypropyl-γ-cyclodextrin, etc.), sulfoalkyl ether cyclodextrin (for example, sulfobutyl ether-α-cyclodextrin, sulfobutyl ether-β-cyclodextrin, sulfobutyl ether-γ-cyclodextrin, etc.), and branched cyclodextrin (for example, maltosyl-α-cyclodextrin, maltosyl-β-cyclodextrin, maltosyl-γ-cyclodextrin, etc.).

Cyclodextrins may be used alone or in arbitrary combination of two or more types. The cyclodextrin to be mixed is preferably hydroxypropyl-β-cyclodextrin or sulfobutyl ether-β-cyclodextrin, and is more preferably sulfobutyl ether-β-cyclodextrin.

The content of such cyclodextrins is not particularly limited, and it is determined, as appropriate, depending on the type thereof, the intended use of an injection, usage, etc. The content of cyclodextrins is preferably 5% to 50% by weight, more preferably 10% to 40% by weight, and further preferably 10% to 30% by weight, based on the total amount of the injection.

In general, in consideration of the ratio of the active ingredient mixed into the preparation, the dose of the pharmaceutical composition of the present invention can be appropriately determined in a wide range, while taking into consideration the age and body weight of an administration target (patient), the type of disease and/or progression status, an administration route, the number of doses (/day), an administration period, etc.

The case of using the pharmaceutical composition of the present invention as a parenteral agent or an oral agent will be specifically described below.

In the case of using the present pharmaceutical composition as a parenteral agent, in general, the dosage form thereof is not limited. In the case of various types of injections, they may be provided, for example, in the state of a unit dose ampoule or a high dose container, or in the state of a lyophilized powder that is to be redissolved in a solution at the time of use. The parenteral agent contains the RP58 protein serving as an active ingredient, and depending on various types of forms, may also contain various types of known excipients or additives in a range that does not impair the effects of the above-described active ingredient. For example, in the case of various types of injections, water, glycerol, propylene glycol, aliphatic polyalcohols such as polyethylene glycol, and the like may be used.

The dose (per day) of the parenteral agent is not limited. For example, in the case of various types of injections, in general, the RP58 protein serving as an active ingredient, etc. can be administered to an application target (a subject, a patient, etc.) at a dose of 0.01 to 1000 mg, 0.05 to 500 mg, or 0.1 to 50 mg per kg of body weight. Otherwise, the dose can be set to be 0.5 to 20 mg, or 1 to 10 mg.

In the case of using as an oral agent, in general, the form thereof is not limited, and any of the aforementioned dosage forms may be applied, or it may also be a dried product that is to be redissolved at the time of use. The oral agent contains the RP58 protein serving as an active ingredient, and depending on various types of forms, may also contain various types of known excipients or additives in a range that does not impair the effects of the above-described active ingredient. Examples of the excipients or additives may include binders (syrup, gum Arabic, gelatin, sorbitol, tragacanth, polyvinylpyrrolidone, etc.), fillers (lactose, sugar, corn starch, potato starch, calcium phosphate, sorbitol, glycine, etc.), lubricants (magnesium stearate, talc, polyethylene glycol, silica, etc.), disintegrants (various types of starches, etc.), and wetting agents (sodium lauryl sulfate, etc.).

With regard to the dose (per day) of the oral agent, in general, the RP58 protein serving as an active ingredient, etc. can be administered to an application target (a subject, a patient, etc.) at a dose of 0.05 to 5000 mg, 0.1 to 1000 mg, or 0.1 to 100 mg per kg of body weight. Otherwise, the dose can be set to be 0.5 to 50 mg, or 1 to 10 mg. The ratio of the active ingredient mixed into the oral agent is not limited, and can be appropriately determined, while taking into consideration the number of doses per day, etc.

Further, the present invention can also provide a pharmaceutical composition for use in the treatment or prevention of brain dysfunction or a disease related thereto, wherein the pharmaceutical composition comprises a nucleic acid encoding the transcription factor RP58, what is called, a gene therapy agent (hereinafter referred to as “the gene therapy agent of the present invention”).

The nucleic acid used in the gene therapy agent of the present invention is not limited, and it may be any of DNA (chromosomal DNA, plasmid DNA, etc.), RNA (mRNA, etc.), peptide nucleic acid (PNA), and oligonucleotide (siRNA, etc.). It may also be a hybrid of any two or more of these.

Specific examples of the nucleic acid encoding the RP58 protein may preferably include, but are not limited to, the following DNAs.

(a1) DNA comprising the nucleotide sequence as set forth in SEQ ID NO: 1.

(b1) RNA consisting of the nucleotide sequence as set forth in SEQ ID NO: 11, 12, 13, 14, or 15.

(c1) DNA consisting of the nucleotide sequence as set forth in SEQ ID NO: 16 or 18.

(d1) DNA or RNA having an identity of 80% or more to the DNA or RNA consisting of the nucleotide sequence having any sequence number described in the above (a1) to (c1), and encoding a protein having the activity of a transcription factor.

(a2) DNA comprising the nucleotide sequence as set forth in SEQ ID NO: 6.

(b2) RNA consisting of the nucleotide sequence as set forth in SEQ ID NO: 20, 21, 22, 23, 24, 25, 26, or 27.

(c2) DNA consisting of the nucleotide sequence as set forth in SEQ ID NO: 28 or 30.

(d2) DNA or RNA having an identity of 80% or more to the DNA or RNA consisting of the nucleotide sequence having any sequence number described in the above (a2) to (c2), and encoding a protein having the activity of a transcription factor.

The DNA described in each of the above (a1) and (a2) is not limited, and may also be DNA consisting of the nucleotide sequence as set forth in SEQ ID NO: 1 or 6.

The DNA or RNA described in each of the above (d1) and (d2) corresponds to a mutant of the DNA or RNA described in each of the above (a1) to (c1) and (a2) to (c2). Such mutant DNA can be prepared according to the site-directed mutagenesis described, for example, in Molecular cloning 4th Edt. Cold Spring Harbor Laboratory Press (2012), etc. Specifically, such mutant DNA can be prepared according to a known method such as a Kunkel method or a Gapped duplex method, using a mutation introduction kit that utilizes site-directed mutagenesis. Preferred examples of the mutation introduction kit may include QuickChange™ Site-Directed Mutagenesis Kit (manufactured by Stratagene), GeneTailor™ Site-Directed Mutagenesis System (manufactured by Invitrogen), and PrimeSTAR (registered trademark) Mutagenesis Basal Kit (manufactured by Takara Bio, Inc.). Alternatively, such mutant DNA, etc. can also be prepared by using the DNA, etc. described in each of the above (a1) to (c1) and (a2) to (c2) as a template, and performing PCR under suitable conditions, employing PCR primers designed to introduce a missense mutation to obtain nucleotides showing a desired amino acid codon. The DNA polymerase used in PCR is not limited, and it is preferably highly precise DNA polymerase. Preferred examples of such DNA polymerase may include Pwo DNA polymerase (Roche Diagnostics), Pfu DNA polymerase (Promega), Platinum Pfx DNA polymerase (Invitrogen), and KOD-plus-polymerase (Toyobo Co., Ltd.) The PCR reaction conditions may be appropriately determined depending on the optimal temperature of the DNA polymerase used, the length and type of the DNA to be synthesized, and the like.

The mutant DNA or RNA described in each of the above (d1) and (d2) can be obtained by performing known hybridization methods such as colony hybridization, plaque hybridization and Southern blot, using, as a probe, the DNA or RNA described in each of the above (a1) to (c1) and (a2) to (c2), or DNA or RNA consisting of a nucleotide sequence complementary thereto, or a fragment thereof, and then obtaining it from a cDNA library or a genomic library. As such a library, a library produced by a known method may be utilized, or a commercially available cDNA library or genomic library may also be utilized, and thus, the library is not limited. Regarding detailed procedures of the hybridization method, Molecular cloning 4th Edt. Cold Spring Harbor Laboratory Press (2012), etc. can be referred, as appropriate.

As described above, the mutant DNA or RNA of each of the above-described (d1) and (d2) preferably consists of a nucleotide sequence having an identity of at least 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more, to the nucleotide sequence of the DNA or RNA of each of the above-described (a1) to (c1) and (a2) to (c2).

The above-described mutant DNA or RNA is DNA encoding a protein having the activity of a transcription factor. Herein, the activity of a transcription factor can also be assayed by a method of evaluating transcriptional function, which is called luciferase assay, or an in vivo function evaluation method involving in utero electroporation.

The gene therapy agent of the present invention may comprise other ingredients other than the above-described DNA or RNA. Examples of such other ingredients may include various types of pharmaceutical ingredients (various types of pharmaceutically acceptable carriers, etc.) that are required depending on the usage (use form) of the gene therapy agent of the present invention. Examples of such pharmaceutical ingredients may include excipients, fillers, thickeners, binders, wetting agents, disintegrants, lubricants, surfactants, dispersants, buffers, preservatives, solubilizers, antiseptics, soothing agents, stabilizers, and tonicity agents, which are commonly used in drug manufacturing.

With regard to administration of the gene therapy agent of the present invention, administration methods include a method of directly administering the gene therapy agent of the present invention by injection and a method of administering a vector into which a gene comprising the above-described DNA has been incorporated. Regarding the above-described vector, various types of known plasmid vectors that can be used as expression vectors, or viral vectors such as an adenovirus vector, an adeno-associated virus vector, a herpesvirus vector, a vaccinia virus vector, a retrovirus vector and a lentivirus vector are used for efficient administration. In addition, commercially available gene transfer kits (for example, product name: AdenoExpress, manufactured by Clontech) can also be used. The present invention also includes a vector (recombinant vector) in which a gene containing a nucleic acid (DNA, etc.) encoding the fusion protein has been incorporated. Furthermore, the present invention also includes a transformant that can be obtained by introducing the aforementioned recombinant vector into a host, so that a gene of interest can be expressed therein. The host is not limited, as long as it can express the gene DNA of the present invention, and examples of the host that can be used herein may include bacteria, yeasts, and animal cells, which are well known in the present technical field.

In general, in consideration of the ratio of the active ingredient mixed into the preparation, the dose of the gene therapy agent of the present invention can be appropriately determined in a wide range, while taking into consideration the age and body weight of an administration target (patient), the type of disease and/or pathological conditions, an administration route, the number of doses, an administration period, etc. For example, when the gene therapy agent of the present invention is administered to an adult patient, the dose of the present gene therapy agent is not limited, but it is preferably approximately 0.1 μg/kg to 1000 mg/kg per body weight of the patient per day, and more preferably approximately 1 μg/kg to 100 mg/kg per body weight of the patient per day.

Besides, the present invention also includes: (i) use of the pharmaceutical composition of the present invention or the gene therapeutic agent of the present invention, and specifically, for example, a method for treating or preventing brain dysfunction or a disease related thereto, or behavioral disorder or a disease related thereto, wherein the method comprises administering an effective amount of the pharmaceutical composition of the present invention or the gene therapeutic agent of the present invention to a test subject (a patient who has or is likely to have brain dysfunction or a disease related thereto or behavioral disorder or a disease related thereto, or such a non-human mammal (e.g., a mouse, a rat, a guinea pig, a rabbit, sheep, a pig, a bovine, a weasel, a cat, a dog, a monkey, etc.); (ii) use of the pharmaceutical composition of the present invention or the gene therapy agent of the present invention for production of a drug for treating or preventing brain dysfunction or a disease related thereto or behavioral disorder or a disease related thereto; and (iii) use of the pharmaceutical composition of the present invention or the gene therapy agent of the present invention for the treatment or prevention of brain dysfunction or a disease related thereto or behavioral disorder or a disease related thereto. In the present invention, specific examples of the treatment of brain dysfunction or a disease related thereto or behavioral disorder or a disease related thereto may include suppression of the progression of the disorder or the disease, the improvement of the prognosis, and/or prevention of the recurrence.

### 5. Kit

Upon the treatment or prevention of brain dysfunction or a disease related thereto or behavioral disorder or a disease related thereto, a kit including the aforementioned pharmaceutical composition of the present invention can be used.

The form of the active ingredient contained in the pharmaceutical composition included in the kit is not limited, and taking into consideration stability (preservation property), the ease of use, and the like, the active ingredient may be prepared in a dissolved state.

The kit can comprise other constitutional elements, as appropriate, as well as the pharmaceutical composition of the present invention.

### 6. Screening Method as Another Aspect, Etc.

The present invention can also provide a screening method as described in (a) or (b) below (i.e., a method of screening for a substance or an environment that improves or reduces cognitive function), in addition to the screening method explained in the above section 3, etc.

(a) A method of screening for a substance that improves or reduces cognitive function, which comprises administering a candidate substance to a wild-type non-human animal, or allowing a candidate substance to come into contact with wild-type animal cells, and then screening the substance that improves or reduces cognitive function, using, as an indicator, the expression level of the transcription factor RP58 in the brain of the non-human animal after the administration or in the animal cells after the contact.

(b) A method of screening for an environment that improves or reduces cognitive function, which comprises breeding a wild-type non-human animal under a candidate environment, or culturing wild-type animal cells under a candidate environment, and then screening the environment that improves or reduces cognitive function, using, as an indicator, the expression level of the transcription factor RP58 in the brain of the non-human animal after the breeding or in the animal cells after the culture.

As described in the Examples of the present application later, when the expression level of the transcription factor RP58 was increased in the neurons of a wild-type non-human animal, the improvement of cognitive function was observed. From these results, a substance or an environmental factor that can increase or decrease the expression level of RP58 may become a substance or an environmental factor that improves or decreases cognitive function. The screening method of the above (a) or (b) was discovered based on these findings, etc.

Examples of the non-human animal that can be used in the methods of the above (a) and (b) may include mammals other than humans, such as a mouse, a rat, a guinea pig, a rabbit, a pig, a weasel, a dog, a cat, a monkey, sheep, a bovine, and a horse. Among these, rodent animals (Rodentia) such as a mouse, a rat and a guinea pig are preferable, and a mouse is more preferable.

Moreover, examples of the animal cells that can be used in the methods of the above (a) and (b) may include cells derived from the above-described non-human animals and human-derived cells. The types of the cells are not limited, and cells of the central nervous system are preferable. Examples of the cells of the central nervous system may include, but are not particularly limited, brain nerve (for example, each nerve of cerebrum, diencephalon, midbrain or cerebellum, and excitatory neurons (presynaptic cells)) system cells, all neurons existing in the central nervous system such as medullary nerve and spinal cord nerve, and glial cells (for example, astrocytes, oligodendrocytes, and microglia).

In the method of the above (a), the above-described candidate substance to be administered or contacted is not limited, and examples of the candidate substance may include naturally or artificially synthesized, various types of peptides, proteins (including enzymes and antibodies), nucleic acids (polynucleotides (DNA (antisense DNA, etc.) and RNA), oligonucleotides (siRNA, shRNA, microRNA, etc.), peptide nucleic acids (PNA), etc.), and low molecular weight, middle molecular weight or high molecular weight organic compounds. Furthermore, various types of drugs or various types of foods, which contain or do not contain the above-exemplified substances, can also be exemplified as candidate substances.

Administration of the candidate substance to a non-human animal can be carried out orally or parenterally, and is not limited. In both cases, a known administration method, known administration conditions and the like can be adopted. Even regarding the applied dose of the candidate substance, the dose can be determined, as appropriate, while taking into consideration the type and condition of a test animal, the type of the candidate substance, etc.

Further, with regard to the contact of the candidate substance with animal cells, a method of allowing the candidate substance to come into contact with the cells or to be absorbed by the cells, such as addition of the candidate substance into a medium or a culture solution containing the cells, or conditions for allowing the candidate substance to come into contact with the cells or to be absorbed by the cells, or the like can be adopted, as appropriate. Also, the amount of the candidate substance used to come into contact with the cells can be determined, as appropriate, taking into consideration the types and conditions of animal cells, the type of the candidate substance, etc.

In the method of the above (b), the above-described candidate environment applied upon the breeding or the culture is not limited, and examples of the candidate environment may include a light environment (the wavelength and/or intensity of an illumination light, etc.), a sound environment (the wavelength and/or intensity of an environmental sound, etc.), a thermal environment (temperature), a gas layer environment (humidity, gas ratio, wind velocity, and air volume), a liquid phase environment (the type and/or concentration of a solute and/or a solvent, gas ratio, flow velocity, flow rate, and viscosity), a solid phase environment (the material, quality, hardness, texture, unevenness, color tone, shape, and size of a cage, a floor, incubation equipment, etc.), a gravity environment (gravity, rotation/stirring, vibration, and acceleration), an electromagnetic field (electric field strength and/or electric flux density, magnetic field strength and/or magnetic flux density, and electromagnetic potential), electromagnetic waves (the wavelength and/or intensity of radio waves, infrared rays, visible rays, ultraviolet rays, X-rays, etc.) and/or radiation (particle radiation such as alpha rays, beta rays, neutron rays, proton rays, heavy ion rays, mesonic rays, etc.), social factors (the number, density, type, gender and age of cells, individuals, fakes, etc. that may cause competition, coexistence, or communication), the content of feed as a nutritional environment (ketone diet, high cholesterol diet, high carbohydrate diet, high fat diet, high vitamin diet, excessive sucrose, etc.), the content of drinking water (addition of vitamin, addition of catechins, anti-inflammatory agent, addition of antibiotic, alcohol, sugar, salt, etc.), administration intervals of feed or drinking water, a stress environment (mother-child separation in various periods from immediately after birth to weaning stage, foster parents, isolated breeding, restraint, overcrowding, mixed breeding with different strains or other species in siblings, new cage movement, insufficient bedding, sleep deprivation, etc.), a rich (excellent) environment (an optimal number of breeding, various toys (turntable, tunnel, etc.), a hiding place, gnawing stick, a sufficient amount of bedding, a nesting material, etc.), the presence or absence of handling and the amount thereof, the presence or absence of administration of drugs (narcotics, stimulants, anti-inflammatory agents, and antibiotics), behavioral experiments, a feeding environment (artificial milk and breast milk), a combination of the above-described candidate environments (complexity of patterns), and environmental factors such as spatiotemporal changes in the candidate environment (frequency and/or the number of repetitions). In addition, applicable conditions for each environmental factor can be appropriately determined, while taking into consideration the types of non-human animals or animal cells, the types of the environmental factors, and the like.

When compared with a non-human animal and non-human animal cells before the use of a candidate substance or application of a candidate environment, or when compared with a non-human animal and non-human animal cells used as controls, to which a candidate substance has not been used or a candidate environment has not been applied, if the expression level of the transcription factor RP58 is significantly increased in the non-human animal and non-human animal cells after the use of the candidate substance or application of the candidate environment, the candidate substance or the candidate environment can be evaluated to be a substance or an environment that improves cognitive function. On the other hand, if the expression level of the transcription factor RP58 is significantly decreased in the non-human animal and non-human animal cells after the use of the candidate substance or application of the candidate environment, the candidate substance or the candidate environment can be evaluated to be a substance or an environment that reduces cognitive function. With regard to the expression level of RP58 in a non-human animal, the expression level of RP58 in brain (in brain cells) may be measured according to various types of conventionally known methods. With regard to the expression level of RP58 in non-human animal cells, the expression level of RP58 in the non-human animal cells may be measured according to various types of conventionally known methods.

The substance or environment improving cognitive function, which can be screened by the methods of the above (a) and (b), can also be used as, for example, those capable of treating or preventing brain dysfunction or a disease related thereto (or a disorder related thereto), as explained in the above section 2. Moreover, the substance or environment reducing cognitive function, which can be screened by the same methods as those mentioned above, can also be used as, for example, those capable of treating or preventing behavioral disorder or a disease related thereto (or a disorder related thereto), as explained in the above section 2.

Accordingly, the present invention can also provide: an agent for improving or reducing cognitive function, comprising the substance obtained by the screening method of the above (a); and further, a method for improving or reducing cognitive function, comprising administering the aforementioned agent for improving or reducing cognitive function to a subject. Besides, for the above-described agent for improving or reducing cognitive function, the explanation of the above section 4. (“Pharmaceutical composition”) can be partially applied, as appropriate.

Moreover, the present invention can also provide a method for improving or reducing cognitive function, comprising providing the environment screened by the method of the above (b) to a subject.

The subject is not particularly limited. The subject (or patient) who wants to improve cognitive function or wants to reduce cognitive function can be arbitrarily adopted. In such a case, a patient who has or is likely to have brain dysfunction or a disease related thereto (or a disorder related thereto), or a patient who has or is likely to have behavioral disorder or a disease related thereto (or a disorder related thereto) can also be used as a subject.

Hereinafter, the present invention will be more specifically described in the following examples. However, the present invention is not limited to these examples.

### Example 1

### [Method]

### <Normal Control Mice >

Wild-type B6 male mice with an age of 4 months old (4 mice), 10 months old (3 mice), and 19 months old (4 mice) were bred under an ordinary environment, and were then used in behavioral experiments.

### <Production of Transgenic Mice (RP58 Expression-Controlled Mouse Models)>

FAST system (Tanaka et al., Biol Psychiatry, 2010) was used. Specifically, first, two types of mice (B6 mice) were produced depending on a difference in the introduction (insertion) site of a loxP-FRT-Neo-STOP-FRT-TetO-loxP sequence (hereinafter referred to as a “STOP sequence”; SEQ ID NO: 32), in which the loxP-FRT-Neo-STOP-FRT-TetO-loxP sequence was introduced into a site located upstream of the translation initiation site of RP58 according to a genetic recombination technology (V1: immediately before the translation initiation site (ATG) of transcription product variant 1; and V2: approximately 140 bp before the translation initiation site (ATG) of transcription product variant 2). Specifically, Exons 1 to 4 (SEQ ID NOs: 7 to 10, successively from Exon 1) are present in the genomic DNA (SEQ ID NO: 6) of mouse RP58. In the case of the above-described V1 mouse, the STOP sequence (which is actually a sequence comprising nucleotides further added to the 5′- and 3′-termini of the STOP sequence (SEQ ID NO: 33)) is inserted into a site directly above the start codon (ATG) in Exon 2 (SEQ ID NO: 8) (i.e., between the 149th nucleotide (T) and the 150th nucleotide (A) of Exon 2). On the other hand, in the above-described V2 mouse, the STOP sequence (which is actually a sequence comprising nucleotides further added to the 5′- and 3′-termini of the STOP sequence (SEQ ID NO: 34)) is inserted into a site located 132 nucleotides upstream of the start codon (ATG) in Exon 3 (SEQ ID NO: 9) (i.e., between the 1130th nucleotide (G) and the 1131st nucleotide (C) of Exon 3). The thus produced two types of mice (V1 and V2) were each mated with Flipases expression mice to produce TetO mice from which the Stop sequence was removed. Furthermore, transsilencer (tTS) expression mice or transactivator (tTA) expression mice were mated with these mice, so as to produce RP58 expression-controlled mouse models (a total of 4 types). As a tTS promoter for RP58 expression decrease models, an Actin promoter was used. As a tTA promoter for expression increase models, a Camk2a promoter was used. In the RP58 expression decrease mouse models, tTS is expressed in almost all cells including brain neurons as a whole. In the RP58 expression increase mouse models, tTA is expressed in mature excitatory neurons. These tTS and tTA bind to tetO in the absence of doxycycline (Dox), so as to cause a decrease or increase in the expression of RP58. In contrast, in the presence of Dox, the tTS and tTA do not bind to tetO, and each exhibit a wild-type phenotype (i.e., a normal expression level of RP58).

As expression decrease models, ActintTS::tetO Rp58 homo and hetero mice were produced, and Dox was then administered thereto until the 21st day after birth (P21; weaning stage), so that the expression level of RP58 was maintained at a normal level. From the 21st day after birth, administration of Dox was suspended, so that tTS was allowed to bind to tetO, and the expression of RP58 was thereby decreased. The same applied to the mice on the 60th day after birth (P60) and thereafter.

As expression increase models, Camk2tTA::tetO Rp58 hetero mice were produced, and then, without administration of doxycycline (Dox), the expression of Rp58 in mature excitatory neurons was increased from the early nascent stage.

### <Quantification of Cognitive Function by Behavioral Test>

4-Month-old, 10-month-old, and 19-month old mice were used as normal control mice. 4- to 5-Month old mice were used as expression decrease mouse models. 4-Month-old and 15-month old mice were used as expression increase mouse models. With reference to the method of Vogel-Ciernia and Wood et al., an “object location recognition test (1 hour memory)” was carried out. Each mouse was habituated in an open field box for 10 minutes. One hour later, two objects having an identical shape were placed in parallel, and the mice were each allowed to search freely for 5 minutes. One hour later, one object was moved to a novel position, and the mice were then allowed to search freely for 5 minutes. The time in which the mice contacted with the novel location object (B) and the familiar location object (A) was calculated with Discrimination Index=(B−A)/(B+A)*100. A plus value indicates that the contact time with the novel location object is long, whereas a minus value indicates that the contact time with the familiar location object is long. The protocols of this object location recognition test are shown in FIG. 1.

Regarding expression increase models, with reference to the method of Denninger et al., a continuous evaluation was carried out also on 10-month old mice according to an “object location recognition +novel object recognition test (3 minutes memory).” Each mouse was habituated in an open field box for 10 minutes. One hour later, objects (A) and (B) each having a different shape were placed in parallel, and the mice were each allowed to search freely for 5 minutes. Three minutes later, the object (B) was moved to a novel location, and the mice were then allowed to search freely for 5 minutes. Further, three minutes later, the familiar object (A) was replaced with a novel object (C), and the mice were then allowed to search freely for 5 minutes. The time in which the mice contacted with the novel location object (B) and the familiar location object (A) was calculated with Discrimination Index=(B−A)/(B+A)*100. The time in which the mice contacted with the novel object (C) and the novel location object (B) was calculated with Discrimination Index=(C−B)/(C+B)*100. A plus value indicates that the contact time with the novel location object or the novel object is long, whereas a minus value indicates that the contact time with the familiar location object or the familiar object is long. The protocols of this object location recognition+novel object recognition test are shown in FIG. 2.

### <Production of Adeno-Associated Virus and Administration Method to Mice >

(i) Production of Plasmid into Which Gene of Interest is Inserted

pAAV-CaMKIIa-eNpHR3.0-EYFP-WPRE (Plasmid #26971, Addgene) was cleaved with the restriction enzymes BamH1/EcoR1, so that eNpHR 3.0-EYFP was removed therefrom, and cMyc-RP58 or GFP was then inserted into the plasmid, so as to produce pAAV-CaMKIIa-cMyc-RP58-WPRE and pAAV-CaMKIIa-GFP-WPRE.

### (ii) Production of Adeno-Associated Virus (AAV) Vector

Three types of plasmid vectors, namely, a plasmid into which a gene of interest had been inserted, an AAV helper plasmid, and an adenovirus helper plasmid were transfected into 293 cells, so as to produce adeno-associated virus vectors with the serotype PHB, comprising the gene of interest therein (AAV-CaMKIIa-cMyc-RP58 and AAV-CaMKIIa-GFP).

(iii) AAV administration Method

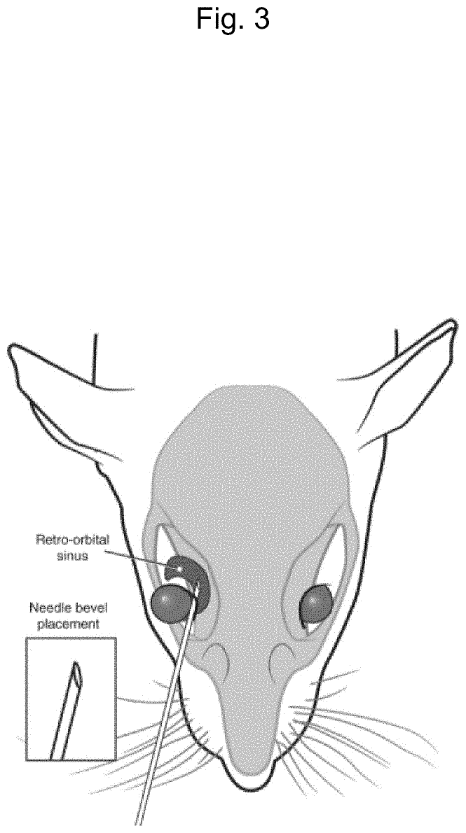

To the orbital sinus of 3-month-old normal control mice and human-type mutation APP homo mice (AD mouse models), a purified adeno-associated virus vector 5×10¹² v.g. (vector genomes) with high purity, together with Evans blue (approximately 1%), was administered using a normal saline as a solvent (FIG. 3; the above-mentioned Non Patent Literature 20: Tal et al. Lab Anim (NY). 2011).

### <Tissue Analysis>

The mice were subjected to perfusion fixation with 4% paraformaldehyde under isoflurane anesthesia. Thereafter, the mice were subjected to post-fixation overnight, and were then subjected to substitution with 20% sucrose and cryostat, so as to produce a frozen section (with a thickness of 50 μm) of the mouse brain tissues. The section was suspended in a phosphate buffered saline, and was then preserved at 4° C. Fluorescent antibody staining was carried out using RP58, cMyc, NeuN, GFP, the amyloid β marker 82E1, and the microglia markers Ibal and CD68.

### [Results and Consideration]

### (1) Behavior Analysis

### <Normal Control Mice >

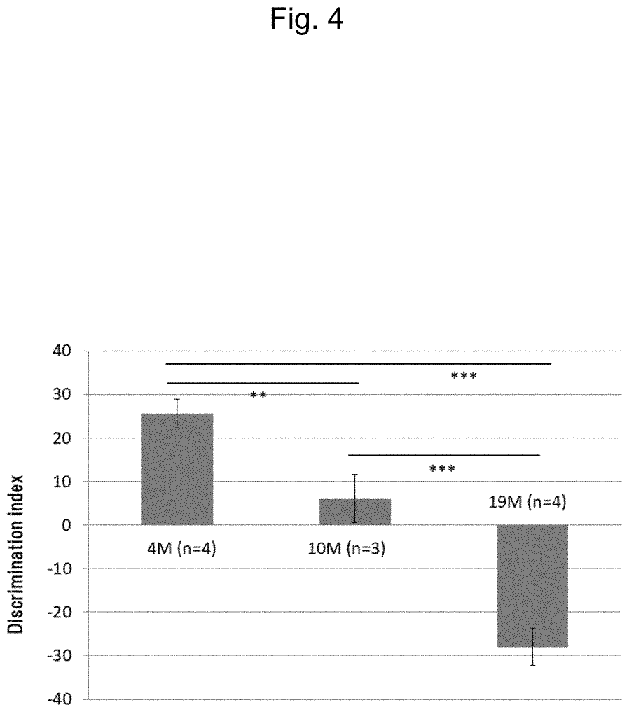

With regard to the value of the discrimination index for evaluation of cognitive function, the value of 4-month-old mice was 25.6 (n=4), the value of 10-month-old mice was 6.1 (n=3), and the value of 19-month-old mice was −28.0 (n=4). The discrimination index was considered to quantitatively show cognitive function, and the discrimination index decreased with aging. It was considered that these results show a reduction in cognitive function due to aging (FIG. 4).

### <Expression Decrease Models>

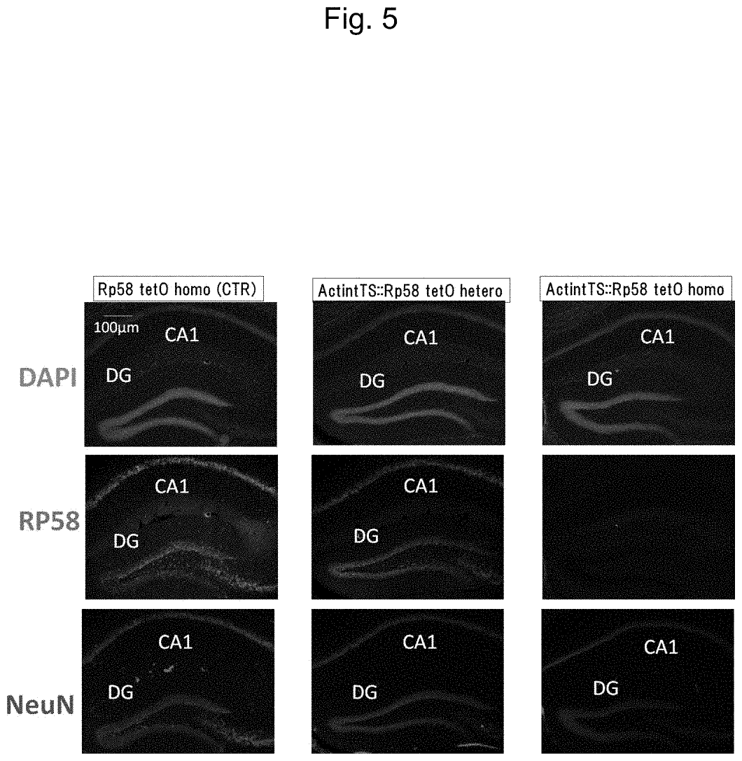

The results obtained by staining ActintTS:: Rp58 tetO homo or hetero mice (5-month-old), in which the expression of RP58 was decreased after P21, with RP58, NeuN, and DAPI are shown (FIG. 5). As control samples, Rp58 tetO homo mice were used. The expression of RP58 was successfully, artificially suppressed.

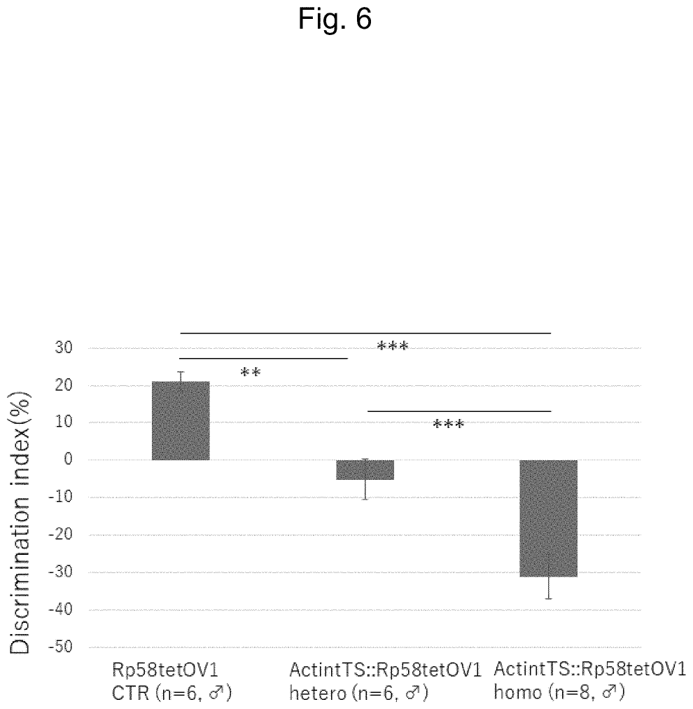

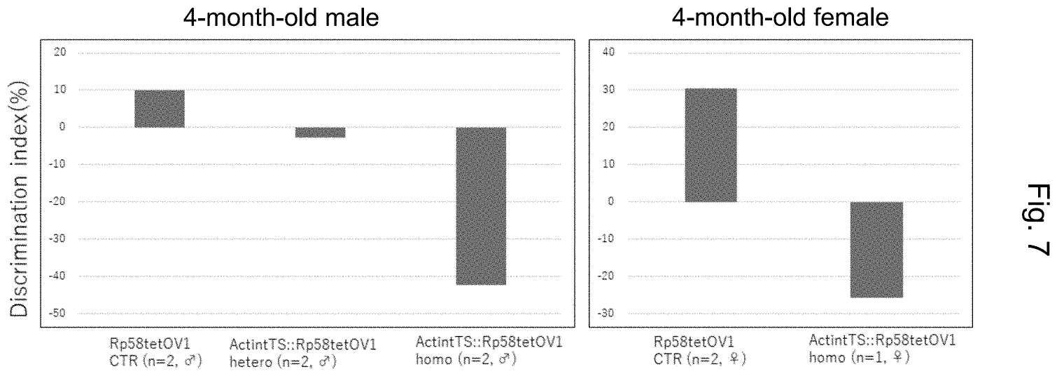

With regard to ActintTS::tetO (V1) Rp58 homo and hetero mice, in which the expression of RP58 had been decreased after P21 and P60, a reduction in cognitive function was found according to an object location recognition test performed on the 4- to 5-month-old mice. A change from the normal level was larger in the homo mice in which a decrease in the expression of RP58 was large, than in the hetero mice, and thus, it was found that cognitive function is controlled dependently on the amount of RP58 (FIGS. 6 and 7).

Therefore, it was demonstrated that a decrease in the expression level of RP58 provokes a reduction in cognitive function.

### <Expression Increase Models>

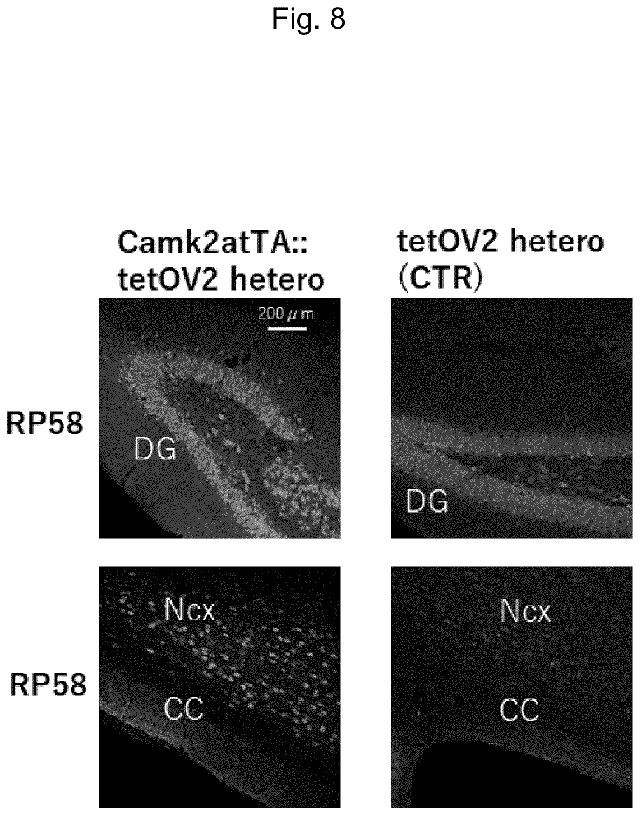

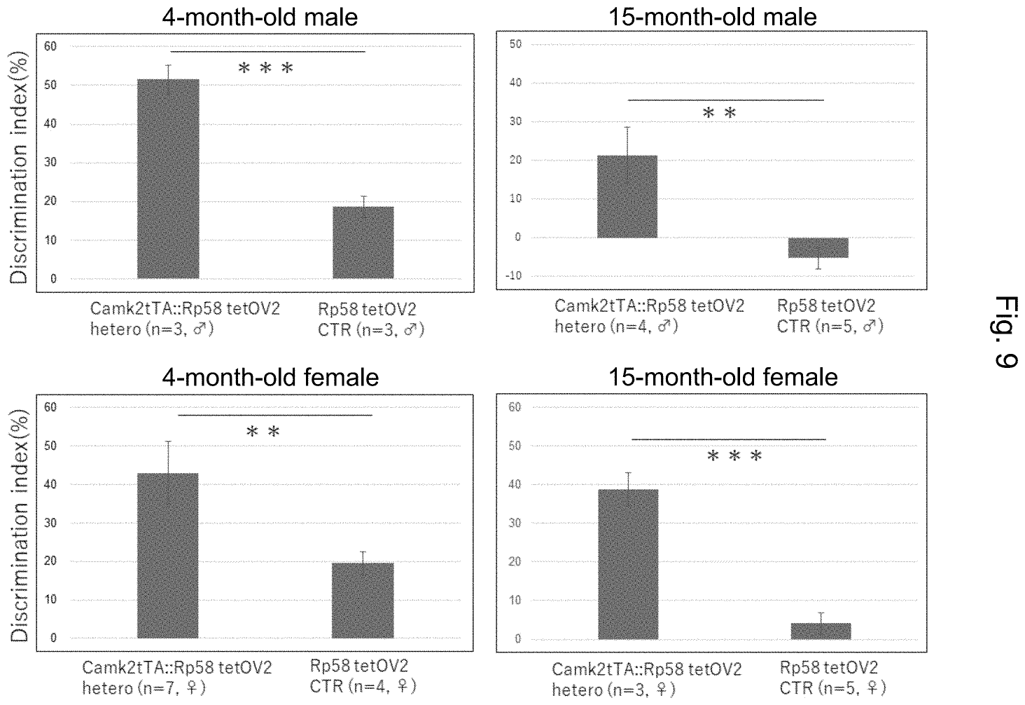

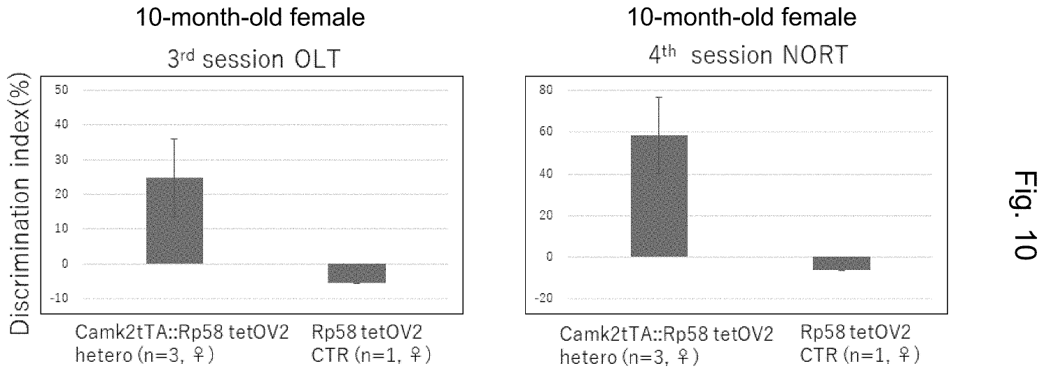

The results obtained by staining with RP58, Camk2tTA:: Rp58 tetO hetero mice (2-month-old), in which the expression of RP58 in mature excitatory neurons was increased from the early nascent stage, are shown (FIG. 8). As control samples, Rp58 tetO hetero mice were used. The expression of RP58 was successfully, artificially increased.

With regard to Camk2tTA::tetO (V2) Rp58 hetero mice, in which the expression of RP58 in mature excitatory neurons had been increased from the early nascent stage, an increase in cognitive function was found on the 4th month according to an object location recognition test (1 hour memory), and further, on the 15th month, a reduction in cognitive function due to aging was suppressed (FIG. 9). Furthermore, on the 10th month, continuous evaluation was carried out according to an object location recognition+novel object recognition test (3 minutes memory). As a result, suppression of a reduction in cognitive function due to aging was observed in the Camk2tTA::tetO Rp58 hetero mice in the two tests (FIG. 10). Therefore, it was demonstrated that a quantitative increase in RP58 induces acceleration in cognitive function, or that a reduction in cognitive function due to aging can be prevented by such a quantitative increase in RP58.

In addition, female Camk2tTA::tetO (V1) Rp58 hetero mice exhibited an action to bite their tail. This action was considered to be self-injury.

### <Expression Increase Models Using AAV Vector>

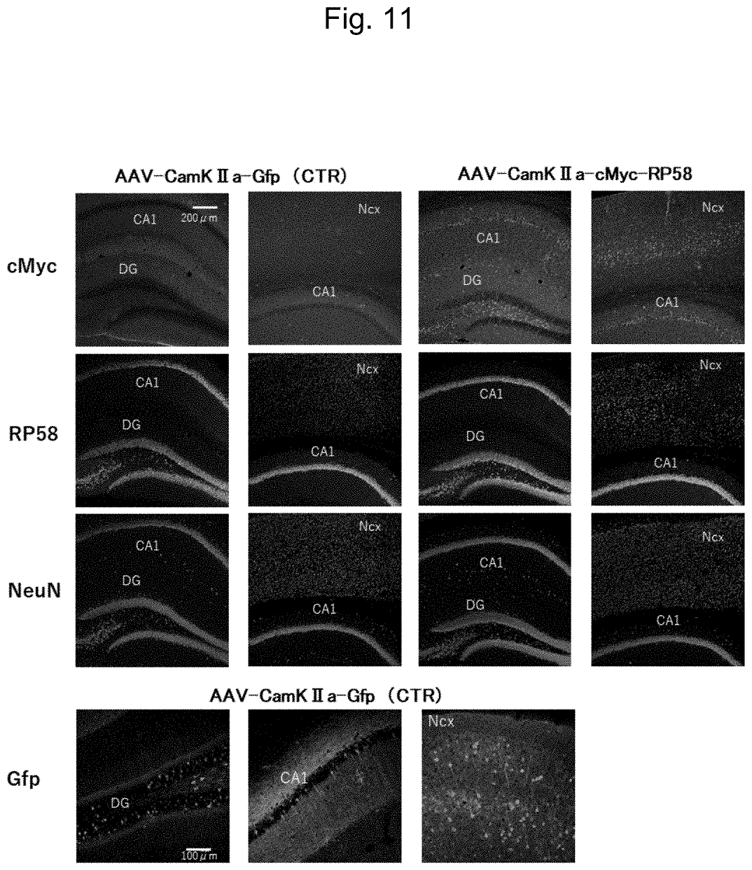

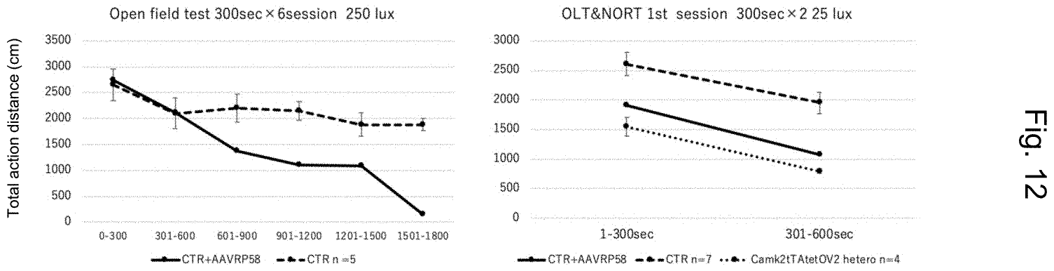

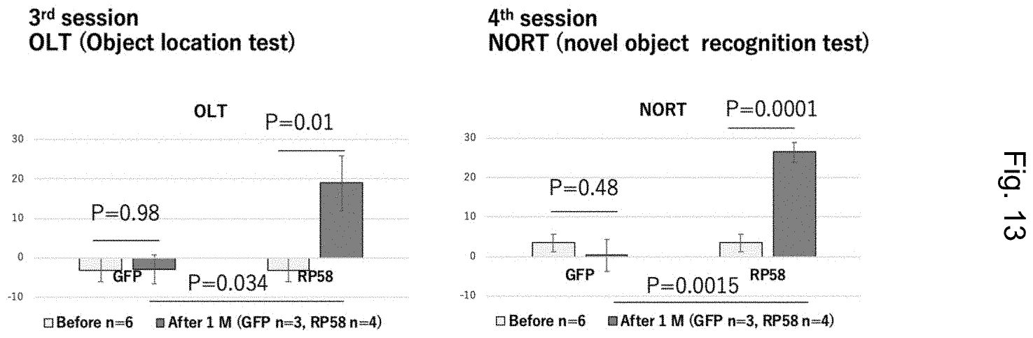

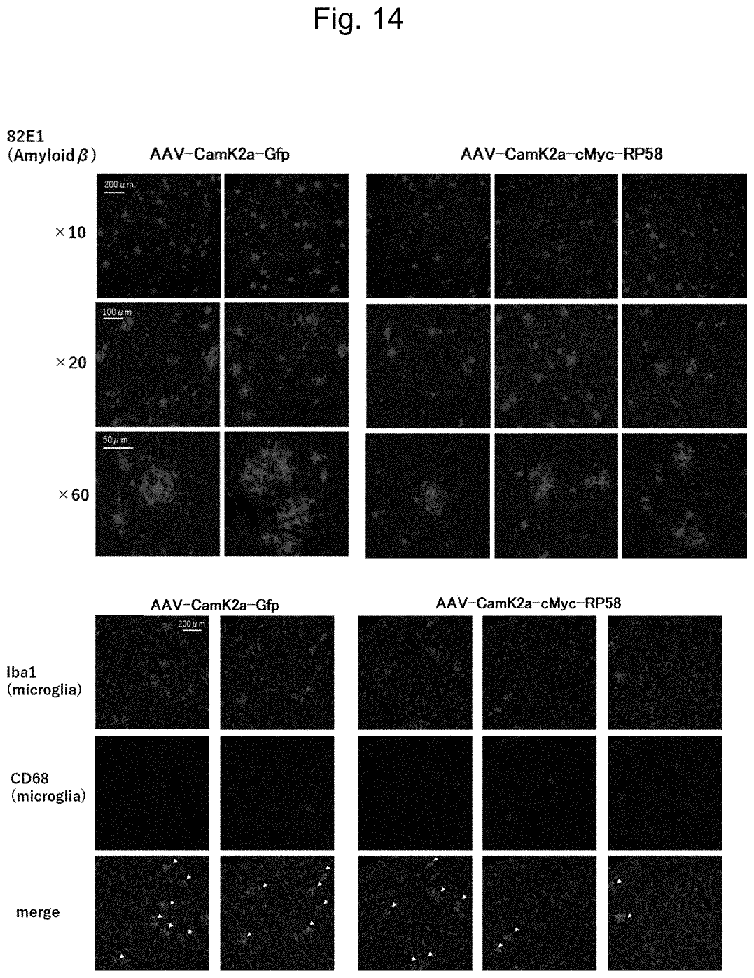

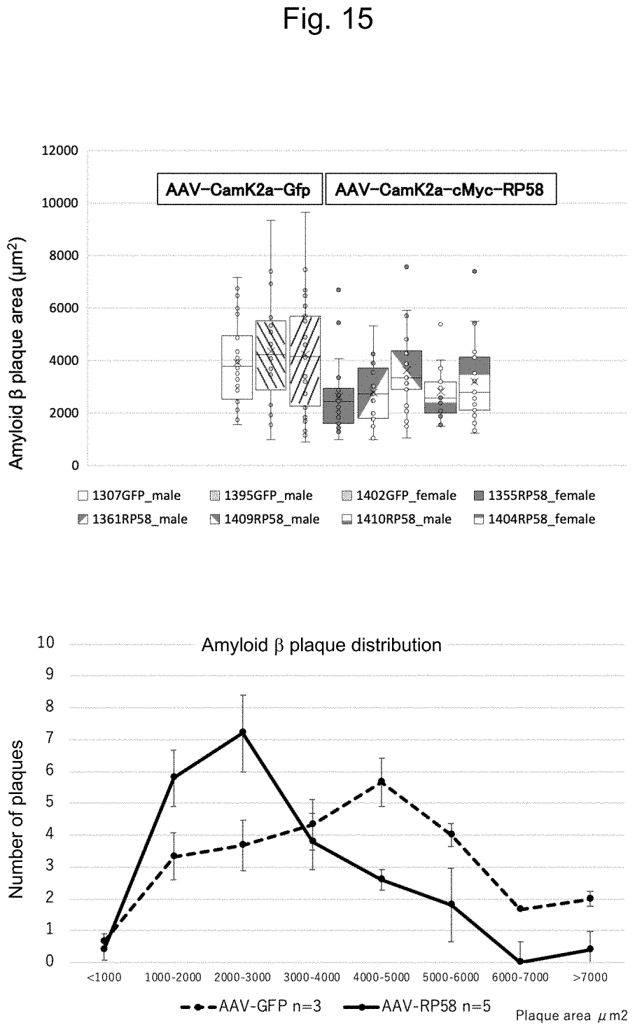

RP58-expressed adeno-associated virus (AAV-CaMKIIa-cMyc-RP58) was intravenously injected into 3-month-old wild-type mice, so that RP58 was overexpressed therein. As a result, cMyc as a marker was detected, and the stainability of RP58 was enhanced. When GFP-expressed AAV was used as a control, the expression of GFP was observed (FIG. 11). Therefore, RP58 could be overexpressed in the neurons of the cerebral cortex and hippocampus.

No change was found in the RP58 overexpression mice according to an object location recognition+novel object recognition test. However, habituation was promoted in an open field test, and one week later, the second contextual searching behavior of the mice was decreased. Thus, acceleration in the habituation was observed (FIG. 12). It is assumed that these results mean the improvement of contextual memory, which is considered to be the improvement of cognitive function. Rodents that were bred under an enrichment environment exhibited acceleration in the above-described habituation (the above-mentioned Non Patent Literature 21: Brenes et al., 2009, the above-mentioned Non Patent Literature 22: Rojas-Carvajal et al., 2018).

### <Effects of Forced Expression of RP58 on Alzheimer's Disease Mouse Models>

It was elucidated that RP58-expressed adeno-associated virus was administered (the above-mentioned Non Patent Literature 24: Matsuzaki et al., Neurosci Lett. 2018) to human-type mutation APP homo mice (AD mouse models; the above-mentioned Non Patent Literature 23: Saito et al., Clin Exp Neuroimmunol. 2018), and as a result, cognitive function was improved (FIG. 13). Specifically, before administration of the virus, the cognitive function of the 3-month-old APP homo mice was reduced according to an object location recognition+novel object recognition test. However, one month after administration of the RP58-expressed virus, the cognitive function of the APP homo mice was returned to a normal level. On the other hand, cognitive function remained low one month after administration of a GFP-expressed virus.

The results obtained by staining with RP58, cMyc, NeuN, GFP, the amyloid β marker 82E1, the microglia markers Ibal and CD68, and the activated-form microglia Iba1+/CD68+ (white arrowhead) in the aforementioned tissue analysis are shown (FIG. 14). As control samples, mice administered with AAV-CaMKIIa-Gfp were used. In an RP58 replenishment group, the size of an amyloid plaque tended to be smaller than the size of a control (FIGS. 14 and 15). These results show that an increase in the amyloid plaques was suppressed by the replenishment of RP68 in 3 months. On the other hand, activation of the microglia tended to be decreased, but a significant change was not observed.

These results demonstrate that the reduced cognitive function of Alzheimer's disease mouse models could be increased by increasing the amount of RP58. In addition, at that time, together with the recovery of the cognitive function, a slight decrease was observed in formation of the amyloid plaques and activation of the microglia. Hence, it was demonstrated that the expression of RP58 in mature excitatory neurons suppresses generation of amyloid plaques and activation of the microglia, and that this can be a factor for the recovery of cognitive function. The level of suppression of the generation of the amyloid plaques and the activation of the microglia due to an increase in RP58 can be demonstrated by increasing the number of cases.

### (2) Conclusion and Future Prospects

A mouse strain capable of increasing or decreasing the amount of RP58 in a stage-specific manner could be constructed using a genetically modified mouse and a viral vector. As a result, it was demonstrated that the amount of RP58 regulates cognitive function, and that a reduction in cognitive function due to aging is caused by a decrease in the expression of RP58.

In particular, by increasing the expression of RP58 in wild-type mice, cognitive function can be accelerated and a reduction in cognitive function due to aging can be suppressed. Hence, a compound capable of increasing the expression of RP58 is screened in cultured cells, or various environmental factors, foods, drugs, etc. are given to wild-type mice, and those capable of increasing the expression of RP58 in the brain are then searched, so that it also becomes possible to discover a method or a substance that promotes acceleration in cognitive function or suppression of a reduction in cognitive function.

Furthermore, the cognitive function of Alzheimer's disease mouse models was improved by replenishment and/or introduction of an RP58 gene. It has been reported that RP58 is decreased in the brain of Alzheimer's disease patients (the above-mentioned Non Patent Literature 25: Castillo et al., 2017). The results of the present example suggest that a reduction in cognitive function found in the brain of Alzheimer's disease patients be related to the expression level of RP58. Accordingly, elucidation of the type of a gene whose expression is suppressed by RP58, in which the suppression of the gene expression leads to suppression of a reduction in cognitive function and the pathological condition of Alzheimer's disease, could be a clue of clarification of the cognitive function and the molecular mechanism of Alzheimer's disease. Moreover, medically, there is a possibility of performing a gene therapy using RP58 on Alzheimer's disease, and at the same time, the searching of a compound capable of increasing the expression of RP58, the searching of a drug for regulating the genetic cascade of a gene located downstream of RP58, and the like are possibly utilized in the discovery of a drug for Alzheimer's disease, and such searching can contribute to the development of a drastic preventive agent or therapeutic agent for Alzheimer's disease, for which a treatment method has not yet been established.

### INDUSTRIAL APPLICABILTY

The transgenic non-human animal capable of increasing or decreasing the expression of the transcription factor RP58 in cells of the central nervous system of the non-human animal in the nascent stage and/or the during and after developmental stage, and the animal model of brain dysfunction or the like, in which the aforementioned non-human animal is used, according to the present invention are extremely useful, in that the present transgenic non-human animal and the present animal model are able to provide a method of screening for a therapeutic or preventive agent against brain dysfunction or the like, a pharmaceutical composition for use in the treatment or prevention of the brain dysfunction or the like, etc.

### Sequence Listing Free Text

SEQ ID NO: 32: Recombinant DNA

SEQ ID NO: 33: Recombinant DNA

SEQ ID NO: 34: Recombinant DNA

1. A transgenic non-human animal capable of increasing or decreasing the expression of a transcription factor RP58 in cells of the central nervous system of the non-human animal in the nascent stage and/or during and after the developmental stage.

2. The transgenic non-human animal according to claim 1, which is capable of increasing or decreasing the expression of the RP58 in cells of the central nervous system of the non-human animal during and after the developmental stage.

3. The transgenic non-human animal according to claim 1, which is capable of increasing or decreasing the expression of the RP58, in a desired period in the nascent stage and/or the during and after developmental stage of the non-human animal.

4. The transgenic non-human animal according to claim 1, which is capable of increasing or decreasing the expression of the RP58 according to a Tet system.

5. The transgenic non-human animal according to claim 4, into which a promoter and a tetracycline-controlled transcriptional activator (tTA) gene linked to the promoter so that the tTA gene can function by the promoter, and a Tet operator (tetO) are introduced, and which is capable of increasing the expression of the RP58 by non-administration of doxycycline.

6. The transgenic non-human animal according to claim 4, into which a promoter and a tetracycline-controlled transcriptional silencer (tTS) gene linked to the promoter so that the tTS gene can function by the promoter, and a Tet operator (tetO) are introduced, and which is capable of decreasing the expression of the RP58 by non-administration of doxycycline.

7. The transgenic non-human animal according to claim 5, wherein the promoter is a Camk2a promoter or an Actin promoter.

8. The transgenic non-human animal according to claim 5, wherein the expression of the RP58 is maintained at a normal level by administration of doxycycline.

9. The transgenic non-human animal according to claim 1, wherein the cells of the central nervous system are brain nervous system cells.

10. The transgenic non-human animal according to claim 1, wherein the non-human animal is a rodent.

11. The transgenic non-human animal according to claim 10, wherein the rodent is a mouse.

12. An animal model of brain dysfunction or a disease related thereto, in which the expression of the transcription factor RP58 is decreased in cells of the central nervous system of the transgenic non-human animal according to claim 1.

13. The animal model according to claim 12, wherein the expression of the RP58 is decreased in cells of the central nervous system of the transgenic non-human animal during and after the developmental stage.

14. The animal model according to claim 12, wherein the brain dysfunction is at least one selected from the group consisting of brain tumor, cognitive dysfunction, developmental disability, intellectual disability, memory impairment, and age-related brain disorder.

15. A method of screening for a therapeutic or preventive agent against brain dysfunction or a disease related thereto, which comprises a step of decreasing the expression of the transcription factor RP58 in cells of the central nervous system of the transgenic non-human animal according to claim 1, a step of administering a candidate substance to the non-human animal in which the expression of the RP58 is decreased, and a step of evaluating the pathological condition of the brain dysfunction or a disease related thereto in the non-human animal after administration of the candidate substance.

16. The screening method according to claim 15, wherein the step of decreasing the expression of the RP58 is a step of decreasing the expression of the RP58 in cells of the central nervous system of the transgenic non-human animal during and after the developmental stage.

17. The screening method according to claim 15, wherein the brain dysfunction is at least one selected from the group consisting of brain tumor, cognitive dysfunction, developmental disability, intellectual disability, memory impairment, and age-related brain disorder.

18. A pharmaceutical composition for use in the treatment or prevention of brain dysfunction or a disease related thereto, wherein the pharmaceutical composition comprises a transcription factor RP58 protein or a nucleic acid encoding the transcription factor RP58.

19. A pharmaceutical composition for use in the treatment or prevention of brain dysfunction or a disease related thereto, wherein the pharmaceutical composition comprises a therapeutic or preventive agent obtained by the screening method according to claim 15.

20. The pharmaceutical composition according to claim 18, wherein the brain dysfunction is at least one selected from the group consisting of brain tumor, cognitive dysfunction, developmental disability, intellectual disability, memory impairment, and age-related brain disorder.

21. A method for treating or preventing brain dysfunction or a disease related thereto, which comprises administering the pharmaceutical composition according to claim 18 to a patient who has or is likely to have brain dysfunction or a disease related thereto.

22. The therapeutic or preventive method according to claim 21, wherein the brain dysfunction is at least one selected from the group consisting of brain tumor, cognitive dysfunction, developmental disability, intellectual disability, memory impairment, and age-related brain disorder.

23. An animal model of behavioral disorder or a disease related thereto, in which the expression of the transcription factor RP58 is increased in cells of the central nervous system of the transgenic non-human animal according to claim 1.

24. The animal model according to claim 23, wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.

25. A method of screening for a therapeutic or preventive agent against behavioral disorder or a disease related thereto, which comprises a step of increasing the expression of the transcription factor RP58 in cells of the central nervous system of the transgenic non-human animal according to claim 1, a step of administering a candidate substance to the non-human animal in which the expression of the RP58 is increased, and a step of evaluating the pathological condition of the behavioral disorder or a disease related thereto in the non-human animal after administration of the candidate sub stance.

26. The screening method according to claim 25, wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.

27. The screening method according to claim 25, wherein the candidate substance is a nucleic acid capable of suppressing the function of a gene encoding RP58.

28. The screening method according to claim 27, wherein the nucleic acid is antisense DNA, siRNA, shRNA, or microRNA.

29. A pharmaceutical composition for use in the treatment or prevention of behavioral disorder or a disease related thereto, wherein the pharmaceutical composition comprises a therapeutic or preventive agent obtained by the screening method according to claim 25.

30. The pharmaceutical composition according to claim 29, wherein the behavioral disorder or a disease related thereto is at least one selected from the group consisting of borderline personality disorder, schizophrenia, depression, bipolar disorder, eating disorder, dissociative disorder, post-traumatic stress syndrome, autism, addiction, and self-injury.

31. A method for treating or preventing behavioral disorder or a disease related thereto, which comprises administering the pharmaceutical composition according to claim 29 to a patient who has or is likely to have behavioral disorder or a disease related thereto.

32. The therapeutic or preventive method according to claim 31, wherein the behavioral disorder or a disease related thereto is at least one selected from the group consisting of borderline personality disorder, schizophrenia, depression, bipolar disorder, eating disorder, dissociative disorder, post-traumatic stress syndrome, addiction, autism, and self-injury.

33. A method of screening for a substance that improves or reduces cognitive function, which comprises administering a candidate substance to a wild-type non-human animal, or allowing a candidate substance to come into contact with wild-type animal cells, and then screening the substance that improves or reduces cognitive function, using, as an indicator, the expression level of the transcription factor RP58 in the brain of the non-human animal after the administration or in the animal cells after the contact.

34. An agent for improving or reducing cognitive function, comprising a substance obtained by the screening method according to claim 33.

35. A method for improving or reducing cognitive function, comprising administering the agent for improving or reducing cognitive function according to claim 34 to a subject.

36. A method of screening for an environment that improves or reduces cognitive function, which comprises breeding a wild-type non-human animal under a candidate environment, or culturing wild-type animal cells under a candidate environment, and then screening the environment that improves or reduces cognitive function, using, as an indicator, the expression level of the transcription factor RP58 in the brain of the non-human animal after the breeding or in the animal cells after the culture.

37. A method for improving or reducing cognitive function, comprising providing the environment screened by the method according to claim 36 to a subject.


SEQUENCE LISTING (36 sequences)

SEQ ID NO: 1 - DNA, 8538 bp, Homo sapiens
gaggaggcggaggacgcggcggcggcggggacgcggtgacttagggccgctccgtgtgagtcccggcgccggcccctggcccctgacctggcgccccctccgcgccccggcaccccggcgggcggggggcgccagctgtgcggccccggccggctgggcggcgcgggcggagggcgggccgggtcgggggcgcccccgcgagtggagttagtttgtgtggttggagctgttggggcggcggggggcggcgcagctgcgggaggcccgggcgcggggacggggccgctgcagctgcggggtcgggcggggtgcgggttcccggctccgcgccgcgcccccgcgggccggtgcagctgcggcgcggggcgggctcccggccggtgcagctgcggtggcgcctctcgggtgcggagggcggcgggctgggggagggggcttgggagcagccgcagctgcagtggcgcccgggacggccgcggctccggggaccggtgcagctgcagtggcgcccgggggagggggccggcgggggagggggccctcccgggctggtgcagttgcccggaggccccgggggctcgcgcgtcgcggcatcggggaagcggtagcaaccactctcctcccgctggctgcttcttacgacttttcttttgttgttgccgtgacccgggtctgttttctcctctgttgtttaaggtgttttcctgctgcaccggctctgcgccctccacctccttccctgtggttttaaccttcctctttccccgtgtgttttatgcacggcgaactacgtaaagatttgcattgcttccccactcggcacccccgcccccacctctctgaaaaacaaaaccccaacctcacaaaacctctatggatccgcggtagcgagacgtgaaggggttgatttgtggagtgggagttgctggcccattgcggtgcccgggactcattaaactgtcactcaccccccaccccactgggtaaatggggtgattaatgtctgatatattggaatgggtcgagggcatttgtggagaataggtggtgtggctgggaagaaattgatctcccgaggatgtctcctggactaagtgttcataattatgtcactcaccgcgaagttggagaaagttaggtttgtgactttgggaaagcgagaaggacaggacggagtcgtcgggttttgttactctttaactctccgccacggctttctaatgggaacgaagtggttatcaccagggctgggacgctggcgcctcaggccccagttccttttctacccctcgccctcccaagagtctgaggagatgtatttgacattcagagatgtcgcagcaccctgcccggtaccggtcagcccagcccgggtcgcgttacagtttcatcagctattagaaaagacccacaaactggctgaaaagttctcaaacttactagacctggcttccccccacacaaagattttcttgtgatcataaacattagagtgacccccccccaaagaaaaaaaagattgttaagggacacagatcaaggggaaggcaaggttctgctttttctgagcagaaagatcagatatgcaggtgcagcattcattttaagtgactggagcaggttgtatcagtttattaattggtctcgttgaactgaagtactttccaggatttcagaaagtgcagtttcagcttgttggtaccgagaattgaaatttgcattgactgtggcagtgtaagaaatttttctgagaaggtagttgtagataagtaattacataagagtgaaataataaggtgaaccagtctgaagtaatagttctgaacaagtctgccaacatttgagacatgtataccattatctcagcagaaaaaaataaaaaagtgcggtaataatgttgaaatgggactggagatgtcaaatgggttggtttaaaaatatccctaaactgctttagaaactaaacgttggagttgaataaaagtgaatctggatttcctgaatagcaccgtggcttgagtgtgctgtcagtatgtaagccagtccaccaacttaagacagaagtgttgctattccataaatacagtagaactccatatgctgagtttttcaatttcttctgttagaaagtttggaaaaaaaggaagactttgaagtattttgtttatactaacagaagagaaaagttctcctaagaaaaagctggtggcaggagtgtagaggagtagcatatggcattaaaaggagcacagctggaggtagcatttccatctgaacatgatgtcagagcagctgacagcctgccatccctcagccttttattctaacacacagacagggagttagtgtgtgcggatctgtggtggagaaggtatctcattcctctctaacatcatctccactttgcatctgtctctcttagtctgctttttttccaccgatgtaacagacctggagccagcaggactcagaggaaaggacttaatcaggtttatgtgtcctaaaggtaggagtagcaagagattagattgagcatatttctgttttggtgttttgtttatttaattttaaaaatcacattacttttctaacccagtttaggaataatatatgaaattgagtattaaatgaacatcttactggagttgaaagtcttgagcttgtttagttttaattttttatcccttttacttgagcgagtaaataactttaatacacaaatatttcaagttttgaaatgttaagaaaggctttaaaatgctgcttttggaaatcacgttggattaaaatgcttttttgttgttcactgctatttagatttgattggggtggtggtggtggggaaatagtaaactggaatccagcattcaaacttatatgagaagattattctagttaagcagctagattgttgtataattaattcctctgtgaaactttattcttcaaaacattgagcagcactactgaaacatacagctgtagtgctgttgaaactgcaggtttttaagaagaagaaaaaaaaaccaaacttttgtccccaggagaatagtttgactggtttcctcatttctctttcaaatatatatttaagagatcttttatttcagcataacaataattatttgcaggagtttttgtgttagcgactaatttagaacttgtagatttgagctgcctgaattggccagacagctgtaatcgcactcctctattcttaatctctttatctgggcagcagctgccatatggccacagtcagctggatcagatgtttataagcttccttcttcgtccctctctgtcctctcagcctcctaatttgatcacggctaccttgtgagctgccctccaatctttatttttagccttctcaaggattaggattgatgttagctgtggggcacttaaagtgattgtattgtaaatcttagtttattctagcagtggcatcttgcagaatgtgtctggggttaggataaagttttagtctgtcttgggcaaagtgtagaagaacaagaagaaaggaagtaatttctgaatattaaattaagtgtatctattcccaaaatgcacttgctgagatgggtggtgtggaggcttgcttctggtcacaggaaaggggaagtcgggtcttctttatttttcaggagtttacccactgcagttgaattctgaccctttgtacatctttgcctttctcctccctccatttcactttccctcatcacaaaacaagggcaatgatgaatatttgcaagaatagaagctagtgggaaaacagaaatatatcaaattatattgaaacaaagtattttctgttggctgaggtaaggcagaaccaggaaaaaaaaaaaggttctctttttctaaaaaggcttaaaaaaaaacgttgaacctgtaaagcttttgagttttatttaagcgctgtatgcctttatttttaaaagtttgaattaatttcaatatgaaactcattttgccttgcaacggatcaaagtttagatgagaattttcggagttcagtaaagcccctctggttcctaatattctgaaagtacagtttctacaaagaaatgtaactacttttttctcagttcgttttgaaaaatctttggatttgttcatagtaaagatgatcttttccatctgttggtgcagctccttctgatctcgtacatgtgtgctatacctgcaaatctgaaggcattaacatctgtttttatacacagggcttttgttgtaacaccctgactttaaaagtaagttgtttgctaatttctggactaattatgcaatttcatttttttttcttgattcagccaaatgtgtgtgtgtgtgtttaaactgtgctttctaagcacagtcaggtagcaaaagtaataaaaaggatggttgaacaagttttcttgtatgttccaggatatgtttgggacttttctttgtttattatatgagttgttccctttgaaattaaagctattttgtaggttttgtgggacataatttgataagtagagttaattaaatttcttctggaagagatctaaattcttattcttagtgagagactgtagttaaaggaaggcttttagaacttgggttcaaggaagatggagatgcgtcggaagctctttggcgggggtgaggaagttcagaaagtgtgcattttccttctggcatttaggtcttgtccgtgtgatttggtggtgcttgggtcataagcctgattaaaattcagggacatgtaccacggcggccaaagcggaattaatttttttatatggggactggagcgctgaaaagttgttcctgaccaggctctaatgagaaattcctctctccccaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaataacaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaa

SEQ ID NO: 2 - DNA, 55 bp, Homo sapiens
gaggaggcggaggacgcggcggcggcggggacgcggtgacttagggccgctccgt

SEQ ID NO: 3 - DNA, 186 bp, Homo sapiens
attctaacacacagacagggagttagtgtgtgcggatctgtggtggagaaggtatctcattcctctctaacatcatctccactttgcatctgtctctcttagtctgctttttttccaccgatgtaacagacctggagccagcaggactcagaggaaaggacttaatcaggtttatgtgtcctaaag

SEQ ID NO: 4 - DNA, 584 bp, Homo sapiens
gttgtttgctaatttctggactaattatgcaatttcatttttttttcttgattcagccaaatgtgtgtgtgtgtgtttaaactgtgctttctaagcacagtcaggtagcaaaagtaataaaaaggatggttgaacaagttttcttgtatgttccaggatatgtttgggacttttctttgtttattatatgagttgttccctttgaaattaaagctattttgtaggttttgtgggacataatttgataagtagagttaattaaatttcttctggaagagatctaaattcttattcttagtgagagactgtagttaaaggaaggcttttagaacttgggttcaaggaagatggagatgcgtcggaagctctttggcgggggtgaggaagttcagaaagtgtgcattttccttctggcatttaggtcttgtccgtgtgatttggtggtgcttgggtcataagcctgattaaaattcagggacatgtaccacggcggccaaagcggaattaatttttttatatggggactggagcgctgaaaagttgttcctgaccaggctctaatgagaaattcctctctccccagg

SEQ ID NO: 5 - DNA, 3688 bp, Homo sapiens
ttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaataacaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaa

SEQ ID NO: 6 - DNA, 8409 bp, Mus musculus
gcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgtgtgagtccccgcgcccgccctgccccgctgccgaccgtgggctcccgccgggcggagttagttagtgtggcggagggcaggcggtgcgggctcgcctcgcctcgcctggccgcgctccccggccggtgcagctgcggcgcggggcggactcggtgcagctgcggcggcgcctcccggtgcggagggcgggcgggcgggcgggggagggggccgggccgccgcagctgcagcgcccgggacggccgcggctccggggccggtgcagctgcagggccgggggagggggagggcgcggcgcccgggttgcggttgccgggaggccccgggggctggcgcgcggcggcggcgacgggggagcggtcgccgcgccgcttcttgcgacttttcttttgttgttgccgcgaccggggtctgttttctcggccgttgtggaaggtgctttcctgctgcaccggctctctgccctgcaccttcttccccgtggtttcacccttcctctttccctgaagtgctttatgcaccgcgaactgcgggaagatttgcatctcctctcccacccctgaacctcacacacacacccggggcaccctgcccccacctctgaaaaacaaaccccctaacctcacagaacctccgagggtccgcggtagcggtggacgggtcgatccgtggagtggatgtcttcgggccattgcggtgcttgggactcattaaactgtcactcacccccaccaccaccccactgggtaaactgggtgattaatgtcggatatagtggaatggggcgagggcatctgtggaaaagagggtgtgtgtgtggcagggaagtaatgtgtctatctgcacagagtgcctcccgaacgagttcagaattgtgtcattcaccgcgaagttagagaaagttaggtttgtgactcgtgagcgagagcagaacacagttggggttttgttctctagctccctgcctctgttttctaagcgagcctaagtggttgtcgcctgggttgggacgcttgtgcctcaggccccaacttcttttttctactctccaacctcccaagggtcggtggagatgtgtctgatattcatagatgtcgcagcaccttgcagggcacccagcgacccagccccggttacattaacattacatcagccattagaaaagatccacaaactgtagccaaaaaaagttcccaaagtctacacttggcctcccaaccctcctggtgatcacaacccctttccccaaagttatagagaccaagggcaagacaaaagtgacttttctgagcaggaagacaagctgtgtggtcaaacaaccattctaagtgactgtagcaggttttgtcagtttattaatttgcctcattgaactaaaatactttccagtatttcagaagtgcagtttgagtttgttgatgctgaaagtggaaatttgcactaacggtggcagtgtgaactattttcctgagaggtagatccagataaattgcttaggtaagagtaaaacaataaggtgaaccacgctgaagtaatagttcttaataagtctgccaacatttcggagatgtctgccattatctccgcaggggaagggggaaaaacatgctgtaataatgttgaaatgggactagagatgtcaaatgggttggtttaaaaatatccctaagctgcctttgaaactagacgttggagttgaataaaagtgaatgtgggtttcctgaatagcaccgagctcgagtgtgctgtcatatgtaagccagtccaccaacttgaggcagaagtgttgcgattccataaatacagtaggactccatatgctgagcttttcaatttcttctgttagaaagtttggggggaaaaaggaagactttgaagtattttgtttatactaacagaagagaaaagttctcctaagaaaaagctggtggcaggagtgtagaggagtagcatatggcattaaaggagcacagctggaggtagcatttccatctgaacatgatgtcagagcagctgacagcctgccatccctcagccttttattctaacacacagacagggagttagtgtgtgcggatctgtggtggagaaggtatctcattcctctccaacatcatctccactttgcatctgtctctcttagtctgctttttttccaccgatgtaacagacctgcagccagcaagactccgagggaaggacttaatcaagtttatgtgtcctaaaggtaggagtagcaggagattaggctgagcatctttgtgtttgggtgttttatttaattttttttaaaatcacattacttttctaatccactttataaataatatgtgaaattgagcatgaagtgatggtcttactggattgaaagtcttgggcttgcttggtttacgattttatttcttctatttgagcaagtaaataactttaatacacaaaaatatttcaagttttcagatggtaagaaacgctctggaatatggcttttggagctcactgtggattaagatgtcttttgttcactgctgtttagattccttttgcttgaggggggcagaaacagtaaactgtctggaatccagctttcaaacttacacgagaagattattctggttaagaagctaggttgtgtaattaattccactgtgaaactttattcttcaaaacattcggcaagcattactgaaagcagcatacagctgtagtgcttttgaaaccggagggtttttgttgttgggttttttttttgttttgttttgttttttgttttttaagaaggaaggaagaaagaaagagaacttttgttcctaggagaatagtttgaccagtttcctcatttctcttttaaatatatatttaagagatcttttatttcaacataacaataattatttgcaggagtttttgtgttagcgactaatttagaacttgtagatgtgagctgcctgaattggccagacagctgtaatcgcactcctctattcttaatctctttatctgggcagcagctgccatatggccacagtcagctggatcagatgtttataagcttccttcttcgtccctctgtcctctcagcctcctaatttgatcacggctaccttgtgagctgccctccaatctttatttttagccctctcaaggattaggattgatgttagctgtagggcacttaaagtgattgtattgtaaatcttggtttattctattggtggtgtctttctttcagaaggtatcggggttaggatgaaagtttcactgtgtctcagcatagtgtggaggagagaagtagtttctgaatagtagcttcatatccatccaagatgcacttgcagaggtagatgttggcgagctctgcctttggtcccaggatggggaagtgggctccgctgatcttaagagagttttagccactacagatgaattttgactgatctttgccttcccttctttcaccctctcccttgccaaaaaacatgggaaagatgcataattggaagaatagaaggtactgggaaaacaaaatagatcaggttattattgaaacaaagtattttctgttggatacctgaggcagagccagaaaaaaaagttttatttattttattttttctaggaaggctttaaaaaatatttaacttacaaagtttttgcacctcattttacagtggtaaaagtttgattaattttaatatgaagttccttttgccctatagcagatcaaagttcagatgaaaatgttcagagttaacagactctctggttgctaatgttgttgaggtagaggatctacaaagaaacaggaccccccccccattcatttttaaaatgtttggatttgttcatagtaaagatgatctttcccatctgtcggtacagttctgctcctctgtgtgtgtagtacctgcagatcttaccgcaccaacatctgttcttatacacagggcttttgttgttgtaacactctgactttaaaagtaagttgtttgctgatttctgcactaattatgcaatttcattttttttccccttcatttcagccaaatgtgtgtgttcagtctgtgctttctaagtagttgggtatcagacgtgttacaaagaatggctggacatgtcttcttgactcttccagaatttgtgtgggactttgcctgatattatgagttctttccctttgaagttaaagctctcttttaggttttgtaggatgtcatttgatgagttgttggttaaatttctggaagaggtctgagttcttgttctaagtcataagctaaaggaagctttaaacttgaccaggagttatgagtggggtgagcaaagccttggtgattgcatttaggttgaatgtgtggcttgggagtccttggggtgtgagcctgatttaaaattcagacatgtaactctccaccactaaggcggatattttttaaagtggcgactagagtctaaattcctgactaggctctaacgagaaactcctctttccccaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaa

SEQ ID NO: 7 - DNA, 245 bp, Mus musculus
aggctgcggccgccgctgggctgctgccatggggagccgttgaaagagtggggccctgtggctcggccgcgcgcctcaggctgccagcgcgggcggggggcggcgggggcgcgcggggcccgggccggggccggcgggcgggcgggggccggcggaggacgcccacaagtccagcggcggcggcggcggcagaggaggaggaggaggaggacgcggcggggacgcggcgactcgggccgctccgt

SEQ ID NO: 8 - DNA, 162 bp, Mus musculus
agtgtgtgcggatctgtggtggagaaggtatctcattcctctccaacatcatctccactttgcatctgtctctcttagtctgctttttttccaccgatgtaacagacctgcagccagcaagactccgagggaaggacttaatcaagtttatgtgtcctaaag

SEQ ID NO: 9 - DNA, 1268 bp, Mus musculus
tcagaaggtatcggggttaggatgaaagtttcactgtgtctcagcatagtgtggaggagagaagtagtttctgaatagtagcttcatatccatccaagatgcacttgcagaggtagatgttggcgagctctgcctttggtcccaggatggggaagtgggctccgctgatcttaagagagttttagccactacagatgaattttgactgatctttgccttcccttctttcaccctctcccttgccaaaaaacatgggaaagatgcataattggaagaatagaaggtactgggaaaacaaaatagatcaggttattattgaaacaaagtattttctgttggatacctgaggcagagccagaaaaaaaagttttatttattttattttttctaggaaggctttaaaaaatatttaacttacaaagtttttgcacctcattttacagtggtaaaagtttgattaattttaatatgaagttccttttgccctatagcagatcaaagttcagatgaaaatgttcagagttaacagactctctggttgctaatgttgttgaggtagaggatctacaaagaaacaggaccccccccccattcatttttaaaatgtttggatttgttcatagtaaagatgatctttcccatctgtcggtacagttctgctcctctgtgtgtgtagtacctgcagatcttaccgcaccaacatctgttcttatacacagggcttttgttgttgtaacactctgactttaaaagtaagttgtttgctgatttctgcactaattatgcaatttcattttttttccccttcatttcagccaaatgtgtgtgttcagtctgtgctttctaagtagttgggtatcagacgtgttacaaagaatggctggacatgtcttcttgactcttccagaatttgtgtgggactttgcctgatattatgagttctttccctttgaagttaaagctctcttttaggttttgtaggatgtcatttgatgagttgttggttaaatttctggaagaggtctgagttcttgttctaagtcataagctaaaggaagctttaaacttgaccaggagttatgagtggggtgagcaaagccttggtgattgcatttaggttgaatgtgtggcttgggagtccttggggtgtgagcctgatttaaaattcagacatgtaactctccaccactaaggcggatattttttaaagtggcgactagagtctaaattcctgactaggctctaacgagaaactcctctttccccag

SEQ ID NO: 10 - DNA, 3676 bp, Mus musculus
gttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataattttatatatatatatatataaataatatatatatatatatatacatatatatacatagatctctatatagttgtggtacggtctaaaagcagtcttgtttcctggaactgaaaagttgggatcttaacttgtttttgcactttagaataatagcatgagaatctcactaatttagcattctgataaaagaaactagagcaagtcacaagtagagagctgtctttcttttgagggttaggccaagttagccaataaaaccttctcaacaaatggttctgtcacaaacgctttcacatggcttacttttgtacacactgcattgtcttgagcgctttccctgtgctccacactgttttgttttgttttgttttcagagtagaaatcccctccagtttattagcctctttatatgtctcaaattgcatgaatattttctggctgttggaaacctgaatgcttttagacccaaatggaaaatttctgaaatgctggattatctatttttaaacaagcagttgacttaaaactttgtgtggcaacttctggttttctgatggtccccagtgagagaaatctgacagtacactgggtcactgggactgtctctgtgaaggtttgcttgagatgaaaaaggatattgcagcttcagcagtgttgaactgtgtgtttaaaatgtgaattactgttcttgtatactgtaattgattacacgggctggggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttgggaaaaaaactggtgttacgaagaacataaatgcactgtctttatttttatttttattttatataatttaaattgacttttcccacgtctttaagtttgaaactgttaagctgaataaaaacttaagctgcaaattgataacttcgctacattacaaggaacatataaatgtttacaaacggcttaaagaattgcatgtgcagtgtgcattcataccacacttgtaattgcacagaacccatgccagctcagagtttaggtgtacacacgtacccagccgagtgtttttagaataactactgcacaaattgacaataggtcataggtcattctctctctccctcctttttgcttcccctttctttttctcccctatttcctttccttccccctaccccctaactcatgaaaagattctatggactgaaaaagccccaggctgaaaggactgaaccgccttgattgacatggggaggggggttagtagactgtgtatcggcagcagaggctgcagcccgatgtgtggttctgatggccagcgcggggcagaagcgttttgcacactgtttgttttcctgtcttgcacttacaaataaggtctatgggagtagcatgggaaacagtggttgtttttccctttttcttttttaattgcttttgtttaaaatttgatcgccttaactactgtaaacatagcctatttttgtgcttaagatactgaatggaaaactccattgtgtgttgctggactgttttggaaatatttggtcaaatgtatgttaatttggctgtaatggcatttaaagcaaacaaacaaacaaacaaacaaagctgtgaaaatggccttggagcattatctttagttacttgaagagtttctagtttttttttttttaatacagtttatgttaaaataatctttattaatctagaggagacaatcaatgtctgtggaagaacggacttttttggattttcttttttaatggtcattgtgagtgattgctctttcttttacttagtttcacattcttcctttgttctaaatcttagactgacatctagctttgacaatcatagtatgttttatttcctgagggggaataacttataatgctgtttagttttgtactattggtgtgttggtgaatttttaaactgtgtgctaactgcaataaattatatgaactgagaa

SEQ ID NO: 11 - RNA, 3791 nt, Homo sapiens
  CDS (70)..(1638)
gaggaggcggaggacgcggcggcggcggggacgcggugacuuagggccgcuccguguuaugaagacaguauggaguuuccagaccauaguagacauuugcuacagugucugagcgagcagagacaccaggguuuucuuugugacugcacuguucuggugggagaugcccaguuccgagcgcaccgagcuguacuggcuucaugcagcauguauuuccaccucuuuuacaaggaccagcuggacaaaagagacauuguucaucugaacagcgacauuguuacagcccccgcuuucgcucuccugcuugaauucauguaugaagggaaacuccaguucaaagacuugcccauugaagacgugcuagcagcugccaguuaucuccacauguaugacauugucaaagucugcaaaaagaagcugaaagagaaagccaccacggaggcagacagcaccaaaaaggaagaagaugcuucaaguuguucggacaaagucgagagucucuccgauggcagcagccacauagcaggcgauuugcccagugaugaagaugaaggagaagaugaaaaauugaacauccugcccagcaaaagggacuuggcggccgagccugggaacauguggaugcgauugcccucagacucagcaggcaucccccaggcuggcggagaggcagagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcuuccugugaugagagugauguuggcacuaaugacuaugacauggaacauagcacugugaaagaaagugugagcacuaauaacaggguacaguaugagccggcccaucuggcuccccugagggaggacucggucuugagggagcuggaccgggaggacaaagccagugaugaugagaugaugaccccagagagcgagcguguccagguggagggaggcauggagagcagucugcuccccuacgucuccaacauccugagccccgcgggccagaucuucaugugcccccugugcaacaaggucuuccccagcccccacauccugcagauccaccugagcacgcacuuccgcgagcaggacggcauccgcagcaagcccgccgccgaugucaacgugcccacgugcucgcugugugggaagacuuucucuugcauguacacccucaagcgccacgagaggacucacucgggggagaagcccuacacaugcacccagugcggcaagagcuuccaguacucgcacaaccugagccgccaugccguggugcacacccgcgagaagccgcacgccugcaaguggugcgagcgcagguucacgcaguccggggaccuguacagacacauucgcaaguuccacugugaguuggugaacuccuugucggucaaaagcgaagcacugagcuugccuacugucagagacuggaccuuagaagauagcucucaagaacuuuggaaauaauuuuauauauauauaaauaauauauauauauauacauauauauaaauagaucucuauauaguugugguacggucuaaaagcagucuuguuuccuggaaauaaaaaguugggauauuaacuuguuuuugcacuuuagaauagcaugagaaucucacuaauuuagcauucugauaaaagaaacuuuagagcaagucagaauagagagguguuuuuccuuugaggggauaggggaaguaagccaauaagaaccuuuuaaacaaaucguccugucacaaaaugcuuucauauggcuuaauuuugucaacacugcauugucuuuugagcucuuuuuucccccccaacaaaguuuuuuuguuuuuuguuuuuuuuuuuaaguagaaauucccuccaguuuuauuagccucuuuauaugucucaaauugcaugaauuuuuucuggcuguuggaaaccugaaugcuuuuagacccaaauggaaaauuucugaaaugcuggauuaucuauuuuuaaacaagcaguugacuuaaaacuuucuguggcaacuucugguuuucugacaguucccagugagagaaaugcugaaaguacacugggaucacugggacacugucuuaugaagguuugcuugggaugaaaaaggauauugcagcuucagcaguguugaacuguguguuuaaaaaugugaauuacuguuauuguauacuguaauugauuacaugggcugggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuuggaaaaaaaaacugguguuaugaagaacguaaaugcacuguuuuuauuuuuauuuuauauaauuuaaauugacuuucccacugucuuuaaguugaaacuguuaagcugaauaaaaacuuaagcugcaaauugauaacuucgcuacauaacaaggaaaauauaaauguuuacaaacagcuuaaagauuugcaugugcagugugcauuuauaacaaacuucuaauugcacaaaacccaugccagcucagaguuuagguguacacauuuacccaguugagcguucuuagaauaacuacugcacaaguugacaauaggucguucucucuuuuuuuuuguuugcucccuuuuucuuuuucuccccuuccuccuuacccucccucccuuacucuccccccccaccaccacccuccacccccaacucaugaaaagauucuauggacugaaaaagccccaggcugaaaggacuggacugccuugauugacauggggaaggggguuaguagacuauguggauugcggcagcagaggcugcagccuaacgugugguuuuaaugaccagcacgcaaggcaaaagcauuuugcacaguguuuguuuuccugucuugcacuuacaaauaaggucuaugggaguagcauggaaaacguuugcuguuuuucccuuuuuuuuuuaauugcuuuuguuuaaaauuugaucgccuuaacuacuguaaacauagccuauuuuugugcuuaagauacugaauggaaaacuccauuguguguugcuggacuguuuuggaaauauuugguuaaauguguguuaauuuggcuguaauggcauuuaaagcaaacaaacaaacaaaaaaagcugugaaaauggccuuggagcauuaucuuuaguuacuugaagaguuucuaguuuuuuuaaaauacaguuuauguuaaaauaauuuuuauuaauuuagagaagacaaucaaugucugugagaaaacggacuuucuuuuggauuuucuuuuuguggucauugugagugauugcuuuuuccuuuucuuaguuucacauucuuccuuuguucuaaaacuuagacugacaucuagcuuugacaaucauaguauguuuuauuuuccugagggggaauaacuuauaaugcuguuuaguuuuguacuauugguguguuggugaauuuuuaaacugugugcuaacugcaauaaauuauaugaacugagaaaaaaaaaaaaaaaaaaaaaaaaaaaaaaaaaaaaaaaaaaaaaaaaa

SEQ ID NO: 12 - RNA, 5905 nt, Homo sapiens
  CDS (2231)..(3799)
guggcgccucucgggugcggagggcggcgggcugggggagggggcuugggagcagccgcagcugcaguggcgcccgggacggccgcggcuccggggaccggugcagcugcaguggcgcccgggggagggggccggcgggggagggggcccucccgggcuggugcaguugcccggaggccccgggggcucgcgcgucgcggcaucggggaagcgguagcaaccacucuccucccgcuggcugcuucuuacgacuuuucuuuuguuguugccgugacccgggucuguuuucuccucuguuguuuaagguguuuuccugcugcaccggcucugcgcccuccaccuccuucccugugguuuuaaccuuccucuuuccccguguguuuuaugcacggcgaacuacguaaagauuugcauugcuuccccacucggcacccccgcccccaccucucugaaaaacaaaaccccaaccucacaaaaccucuauggauccgcgguagcgagacgugaagggguugauuuguggagugggaguugcuggcccauugcggugcccgggacucauuaaacugucacucaccccccaccccacuggguaaauggggugauuaaugucugauauauuggaaugggucgagggcauuuguggagaauaggugguguggcugggaagaaauugaucucccgaggaugucuccuggacuaaguguucauaauuaugucacucaccgcgaaguuggagaaaguuagguuugugacuuugggaaagcgagaaggacaggacggagucgucggguuuuguuacucuuuaacucuccgccacggcuuucuaaugggaacgaagugguuaucaccagggcugggacgcuggcgccucaggccccaguuccuuuucuaccccucgcccucccaagagucugaggagauguauuugacauucagagaugucgcagcacccugcccgguaccggucagcccagcccgggucgcguuacaguuucaucagcuauuagaaaagacccacaaacuggcugaaaaguucucaaacuuacuagaccuggcuuccccccacacaaagauuuucuugugaucauaaacauuagagugacccccccccaaagaaaaaaaagauuguuaagggacacagaucaaggggaaggcaagguucugcuuuuucugagcagaaagaucagauaugcaggugcagcauucauuuuaagugacuggagcagguuguaucaguuuauuaauuggucucguugaacugaaguacuuuccaggauuucagaaagugcaguuucagcuuguugguaccgagaauugaaauuugcauugacuguggcaguguaagaaauuuuucugagaagguaguuguagauaaguaauuacauaagagugaaauaauaaggugaaccagucugaaguaauaguucugaacaagucugccaacauuugagacauguauaccauuaucucagcagaaaaaaauaaaaaagugcgguaauaauguugaaaugggacuggagaugucaaauggguugguuuaaaaauaucccuaaacugcuuuagaaacuaaacguuggaguugaauaaaagugaaucuggauuuccugaauagcaccguggcuugagugugcugucaguauguaagccaguccaccaacuuaagacagaaguguugcuauuccauaaauacaguagaacuccauaugcugaguuuuucaauuucuucuguuagaaaguuuggaaaaaaaggaagacuuugaaguauuuuguuuauacuaacagaagagaaaaguucuccuaagaaaaagcugguggcaggaguguagaggaguagcauauggcauuaaaaggagcacagcuggagguagcauuuccaucugaacaugaugucagagcagcugacagccugccaucccucagccuuuuauucuaacacacagacagggaguuagugugugcggaucugugguggagaagguaucucauuccucucuaacaucaucuccacuuugcaucugucucucuuagucugcuuuuuuuccaccgauguaacagaccuggagccagcaggacucagaggaaaggacuuaaucagguuuauguguccuaaagcuccuucugaucucguacaugugugcuauaccugcaaaucugaaggcauuaacaucuguuuuuauacacagggcuuuuguuguaacacccugacuuuaaaaguuaugaagacaguauggaguuuccagaccauaguagacauuugcuacagugucugagcgagcagagacaccaggguuuucuuugugacugcacuguucuggugggagaugcccaguuccgagcgcaccgagcuguacuggcuucaugcagcauguauuuccaccucuuuuacaaggaccagcuggacaaaagagacauuguucaucugaacagcgacauuguuacagcccccgcuuucgcucuccugcuugaauucauguaugaagggaaacuccaguucaaagacuugcccauugaagacgugcuagcagcugccaguuaucuccacauguaugacauugucaaagucugcaaaaagaagcugaaagagaaagccaccacggaggcagacagcaccaaaaaggaagaagaugcuucaaguuguucggacaaagucgagagucucuccgauggcagcagccacauagcaggcgauuugcccagugaugaagaugaaggagaagaugaaaaauugaacauccugcccagcaaaagggacuuggcggccgagccugggaacauguggaugcgauugcccucagacucagcaggcaucccccaggcuggcggagaggcagagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcuuccugugaugagagugauguuggcacuaaugacuaugacauggaacauagcacugugaaagaaagugugagcacuaauaacaggguacaguaugagccggcccaucuggcuccccugagggaggacucggucuugagggagcuggaccgggaggacaaagccagugaugaugagaugaugaccccagagagcgagcguguccagguggagggaggcauggagagcagucugcuccccuacgucuccaacauccugagccccgcgggccagaucuucaugugcccccugugcaacaaggucuuccccagcccccacauccugcagauccaccugagcacgcacuuccgcgagcaggacggcauccgcagcaagcccgccgccgaugucaacgugcccacgugcucgcugugugggaagacuuucucuugcauguacacccucaagcgccacgagaggacucacucgggggagaagcccuacacaugcacccagugcggcaagagcuuccaguacucgcacaaccugagccgccaugccguggugcacacccgcgagaagccgcacgccugcaaguggugcgagcgcagguucacgcaguccggggaccuguacagacacauucgcaaguuccacugugaguuggugaacuccuugucggucaaaagcgaagcacugagcuugccuacugucagagacuggaccuuagaagauagcucucaagaacuuuggaaauaauuuuauauauauauaaauaauauauauauauauacauauauauaaauagaucucuauauaguugugguacggucuaaaagcagucuuguuuccuggaaauaaaaaguugggauauuaacuuguuuuugcacuuuagaauagcaugagaaucucacuaauuuagcauucugauaaaagaaacuuuagagcaagucagaauagagagguguuuuuccuuugaggggauaggggaaguaagccaauaagaaccuuuuaaacaaaucguccugucacaaaaugcuuucauauggcuuaauuuugucaacacugcauugucuuuugagcucuuuuuucccccccaacaaaguuuuuuuguuuuuuguuuuuuuuuuuaaguagaaauucccuccaguuuuauuagccucuuuauaugucucaaauugcaugaauuuuuucuggcuguuggaaaccugaaugcuuuuagacccaaauggaaaauuucugaaaugcuggauuaucuauuuuuaaacaagcaguugacuuaaaacuuucuguggcaacuucugguuuucugacaguucccagugagagaaaugcugaaaguacacugggaucacugggacacugucuuaugaagguuugcuugggaugaaaaaggauauugcagcuucagcaguguugaacuguguguuuaaaaaugugaauuacuguuauuguauacuguaauugauuacaugggcugggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauaacaccauuuggaaaaaaaaacugguguuaugaagaacguaaaugcacuguuuuuauuuuuauuuuauauaauuuaaauugacuuucccacugucuuuaaguugaaacuguuaagcugaauaaaaacuuaagcugcaaauugauaacuucgcuacauaacaaggaaaauauaaauguuuacaaacagcuuaaagauuugcaugugcagugugcauuuauaacaaacuucuaauugcacaaaacccaugccagcucagaguuuagguguacacauuuacccaguugagcguucuuagaauaacuacugcacaaguugacaauaggucguucucucuuuuuuuuuguuugcucccuuuuucuuuuucuccccuuccuccuuacccucccucccuuacucuccccccccaccaccacccuccacccccaacucaugaaaagauucuauggacugaaaaagccccaggcugaaaggacuggacugccuugauugacauggggaaggggguuaguagacuauguggauugcggcagcagaggcugcagccuaacgugugguuuuaaugaccagcacgcaaggcaaaagcauuuugcacaguguuuguuuuccugucuugcacuuacaaauaaggucuaugggaguagcauggaaaacguuugcuguuuuucccuuuuuuuuuuaauugcuuuuguuuaaaauuugaucgccuuaacuacuguaaacauagccuauuuuugugcuuaagauacugaauggaaaacuccauuguguguugcuggacuguuuuggaaauauuugguuaaauguguguuaauuuggcuguaauggcauuuaaagcaaacaaacaaacaaaaaaagcugugaaaauggccuuggagcauuaucuuuaguuacuugaagaguuucuaguuuuuuuaaaauacaguuuauguuaaaauaauuuuuauuaauuuagagaagacaaucaaugucugugagaaaacggacuuucuuuuggauuuucuuuuuguggucauugugagugauugcuuuuuccuuuucuuaguuucacauucuuccuuuguucuaaaacuuagacugacaucuagcuuugacaaucauaguauguuuuauuuuccugagggggaauaacuuauaaugcuguuuaguuuuguacuauugguguguuggugaauuuuuaaacugugugcuaacugcaauaaauuauaugaacugagaa

SEQ ID NO: 13 - RNA, 3922 nt, Homo sapiens
  CDS (174)..(1769)
auucuaacacacagacagggaguuagugugugcggaucugugguggagaagguaucucauuccucucuaacaucaucuccacuuugcaucugucucucuuagucugcuuuuuuuccaccgauguaacagaccuggagccagcaggacucagaggaaaggacuuaaucagguuuauguguccuaaagguuaugaagacaguauggaguuuccagaccauaguagacauuugcuacagugucugagcgagcagagacaccaggguuuucuuugugacugcacuguucuggugggagaugcccaguuccgagcgcaccgagcuguacuggcuucaugcagcauguauuuccaccucuuuuacaaggaccagcuggacaaaagagacauuguucaucugaacagcgacauuguuacagcccccgcuuucgcucuccugcuugaauucauguaugaagggaaacuccaguucaaagacuugcccauugaagacgugcuagcagcugccaguuaucuccacauguaugacauugucaaagucugcaaaaagaagcugaaagagaaagccaccacggaggcagacagcaccaaaaaggaagaagaugcuucaaguuguucggacaaagucgagagucucuccgauggcagcagccacauagcaggcgauuugcccagugaugaagaugaaggagaagaugaaaaauugaacauccugcccagcaaaagggacuuggcggccgagccugggaacauguggaugcgauugcccucagacucagcaggcaucccccaggcuggcggagaggcagagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcuuccugugaugagagugauguuggcacuaaugacuaugacauggaacauagcacugugaaagaaagugugagcacuaauaacaggguacaguaugagccggcccaucuggcuccccugagggaggacucggucuugagggagcuggaccgggaggacaaagccagugaugaugagaugaugaccccagagagcgagcguguccagguggagggaggcauggagagcagucugcuccccuacgucuccaacauccugagccccgcgggccagaucuucaugugcccccugugcaacaaggucuuccccagcccccacauccugcagauccaccugagcacgcacuuccgcgagcaggacggcauccgcagcaagcccgccgccgaugucaacgugcccacgugcucgcugugugggaagacuuucucuugcauguacacccucaagcgccacgagaggacucacucgggggagaagcccuacacaugcacccagugcggcaagagcuuccaguacucgcacaaccugagccgccaugccguggugcacacccgcgagaagccgcacgccugcaaguggugcgagcgcagguucacgcaguccggggaccuguacagacacauucgcaaguuccacugugaguuggugaacuccuugucggucaaaagcgaagcacugagcuugccuacugucagagacuggaccuuagaagauagcucucaagaacuuuggaaauaauuuuauauauauauaaauaauauauauauauauacauauauauaaauagaucucuauauaguugugguacggucuaaaagcagucuuguuuccuggaaauaaaaaguugggauauuaacuuguuuuugcacuuuagaauagcaugagaaucucacuaauuuagcauucugauaaaagaaacuuuagagcaagucagaauagagagguguuuuuccuuugaggggauaggggaaguaagccaauaagaaccuuuuaaacaaaucguccugucacaaaaugcuuucauauggcuuaauuuugucaacacugcauugucuuuugagcucuuuuuucccccccaacaaaguuuuuuuguuuuuuguuuuuuuuuuuaaguagaaauucccuccaguuuuauuagccucuuuauaugucucaaauugcaugaauuuuuucuggcuguuggaaaccugaaugcuuuuagacccaaauggaaaauuucugaaaugcuggauuaucuauuuuuaaacaagcaguugacuuaaaacuuucuguggcaacuucugguuuucugacaguucccagugagagaaaugcugaaaguacacugggaucacugggacacugucuuaugaagguuugcuugggaugaaaaaggauauugcagcuucagcaguguugaacuguguguuuaaaaaugugaauuacuguuauuguauacuguaauugauuacaugggcugggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuuggaaaaaaaaacugguguuaugaagaacguaaaugcacuguuuuuauuuuuauuuuauauaauuuaaauugacuuucccacugucuuuaaguugaaacuguuaagcugaauaaaaacuuaagcugcaaauugauaacuucgcuacauaacaaggaaaauauaaauguuuacaaacagcuuaaagauuugcaugugcagugugcauuuauaacaaacuucuaauugcacaaaacccaugccagcucagaguuuagguguacacauuuacccaguugagcguucuuagaauaacuacugcacaaguugacaauaggucguucucucuuuuuuuuuguuugcucccuuuuucuuuuucuccccuuccuccuuacccucccucccuuacucuccccccccaccaccacccuccacccccaacucaugaaaagauucuauggacugaaaaagccccaggcugaaaggacuggacugccuugauugacauggggaaggggguuaguagacuauguggauugcggcagcagaggcugcagccuaacgugugguuuuaaugaccagcacgcaaggcaaaagcauuuugcacaguguuuguuuuccugucuugcacuuacaaauaaggucuaugggaguagcauggaaaacguuugcuguuuuucccuuuuuuuuuuaauugcuuuuguuuaaaauuugaucgccuuaacuacuguaaacauagccuauuuuugugcuuaagauacugaauggaaaacuccauuguguguugcuggacuguuuuggaaauauuugguuaaauguguguuaauuuggcuguaauggcauuuaaagcaaacaaacaaacaaaaaaagcugugaaaauggccuuggagcauuaucuuuaguuacuugaagaguuucuaguuuuuuuaaaauacaguuuauguuaaaauaauuuuuauuaauuuagagaagacaaucaaugucugugagaaaacggacuuucuuuuggauuuucuuuuuguggucauugugagugauugcuuuuuccuuuucuuaguuucacauucuuccuuuguucuaaaacuuagacugacaucuagcuuugacaaucauaguauguuuuauuuuccugagggggaauaacuuauaaugcuguuuaguuuuguacuauugguguguuggugaauuuuuaaacugugugcuaacugcaauaaauuauaugaacugagaaaaaaaaaaaaaaaaaaaaaaaaaaaaaaaaaaaaaaaaaaaaaaaaa

SEQ ID NO: 14 - RNA, 3805 nt, Homo sapiens
  CDS (131)..(1699)
agucugcuuuuuuuccaccgauguaacagaccuggagccagcaggacucagaggaaaggacuuaaucagguuuauguguccuaaagggcuuuuguuguaacacccugacuuuaaaaguuaugaagacaguauggaguuuccagaccauaguagacauuugcuacagugucugagcgagcagagacaccaggguuuucuuugugacugcacuguucuggugggagaugcccaguuccgagcgcaccgagcuguacuggcuucaugcagcauguauuuccaccucuuuuacaaggaccagcuggacaaaagagacauuguucaucugaacagcgacauuguuacagcccccgcuuucgcucuccugcuugaauucauguaugaagggaaacuccaguucaaagacuugcccauugaagacgugcuagcagcugccaguuaucuccacauguaugacauugucaaagucugcaaaaagaagcugaaagagaaagccaccacggaggcagacagcaccaaaaaggaagaagaugcuucaaguuguucggacaaagucgagagucucuccgauggcagcagccacauagcaggcgauuugcccagugaugaagaugaaggagaagaugaaaaauugaacauccugcccagcaaaagggacuuggcggccgagccugggaacauguggaugcgauugcccucagacucagcaggcaucccccaggcuggcggagaggcagagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcuuccugugaugagagugauguuggcacuaaugacuaugacauggaacauagcacugugaaagaaagugugagcacuaauaacaggguacaguaugagccggcccaucuggcuccccugagggaggacucggucuugagggagcuggaccgggaggacaaagccagugaugaugagaugaugaccccagagagcgagcguguccagguggagggaggcauggagagcagucugcuccccuacgucuccaacauccugagccccgcgggccagaucuucaugugcccccugugcaacaaggucuuccccagcccccacauccugcagauccaccugagcacgcacuuccgcgagcaggacggcauccgcagcaagcccgccgccgaugucaacgugcccacgugcucgcugugugggaagacuuucucuugcauguacacccucaagcgccacgagaggacucacucgggggagaagcccuacacaugcacccagugcggcaagagcuuccaguacucgcacaaccugagccgccaugccguggugcacacccgcgagaagccgcacgccugcaaguggugcgagcgcagguucacgcaguccggggaccuguacagacacauucgcaaguuccacugugaguuggugaacuccuugucggucaaaagcgaagcacugagcuugccuacugucagagacuggaccuuagaagauagcucucaagaacuuuggaaauaauuuuauauauauauaaauaauauauauauauauacauauauauaaauagaucucuauauaguugugguacggucuaaaagcagucuuguuuccuggaaauaaaaaguugggauauuaacuuguuuuugcacuuuagaauagcaugagaaucucacuaauuuagcauucugauaaaagaaacuuuagagcaagucagaauagagagguguuuuuccuuugaggggauaggggaaguaagccaauaagaaccuuuuaaacaaaucguccugucacaaaaugcuuucauauggcuuaauuuugucaacacugcauugucuuuugagcucuuuuuucccccccaacaaaguuuuuuuguuuuuuguuuuuuuuuuuaaguagaaauucccuccaguuuuauuagccucuuuauaugucucaaauugcaugaauuuuuucuggcuguuggaaaccugaaugcuuuuagacccaaauggaaaauuucugaaaugcuggauuaucuauuuuuaaacaagcaguugacuuaaaacuuucuguggcaacuucugguuuucugacaguucccagugagagaaaugcugaaaguacacugggaucacugggacacugucuuaugaagguuugcuugggaugaaaaaggauauugcagcuucagcaguguugaacuguguguuuaaaaaugugaauuacuguuauuguauacuguaauugauuacaugggcugggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauaacaccauuuggaaaaaaaaacugguguuaugaagaacguaaaugcacuguuuuuauuuuuauuuuauauaauuuaaauugacuuucccacugucuuuaaguugaaacuguuaagcugaauaaaaacuuaagcugcaaauugauaacuucgcuacauaacaaggaaaauauaaauguuuacaaacagcuuaaagauuugcaugugcagugugcauuuauaacaaacuucuaauugcacaaaacccaugccagcucagaguuuagguguacacauuuacccaguugagcguucuuagaauaacuacugcacaaguugacaauaggucguucucucuuuuuuuuuguuugcucccuuuuucuuuuucuccccuuccuccuuacccucccucccuuacucuccccccccaccaccacccuccacccccaacucaugaaaagauucuauggacugaaaaagccccaggcugaaaggacuggacugccuugauugacauggggaaggggguuaguagacuauguggauugcggcagcagaggcugcagccuaacgugugguuuuaaugaccagcacgcaaggcaaaagcauuuugcacaguguuuguuuuccugucuugcacuuacaaauaaggucuaugggaguagcauggaaaacguuugcuguuuuucccuuuuuuuuuuaauugcuuuuguuuaaaauuugaucgccuuaacuacuguaaacauagccuauuuuugugcuuaagauacugaauggaaaacuccauuguguguugcuggacuguuuuggaaauauuugguuaaauguguguuaauuuggcuguaauggcauuuaaagcaaacaaacaaacaaaaaaagcugugaaaauggccuuggagcauuaucuuuaguuacuugaagaguuucuaguuuuuuuaaaauacaguuuauguuaaaauaauuuuuauuaauuuagagaagacaaucaaugucugugagaaaacggacuuucuuuuggauuuucuuuuuguggucauugugagugauugcuuuuuccuuuucuuaguuucacauucuuccuuuguucuaaaacuuagacugacaucuagcuuugacaaucauaguauguuuuauuuuccugagggggaauaacuuauaaugcuguuuaguuuuguacuauugguguguuggugaauuuuuaaacugugugcuaacugcaauaaauuauaugaacugagaa

SEQ ID NO: 15 - RNA, 4319 nt, Homo sapiens
  CDS (598)..(2166)
guuguuugcuaauuucuggacuaauuaugcaauuucauuuuuuuuucuugauucagccaaauguguguguguguguuuaaacugugcuuucuaagcacagucagguagcaaaaguaauaaaaaggaugguugaacaaguuuucuuguauguuccaggauauguuugggacuuuucuuuguuuauuauaugaguuguucccuuugaaauuaaagcuauuuuguagguuuugugggacauaauuugauaaguagaguuaauuaaauuucuucuggaagagaucuaaauucuuauucuuagugagagacuguaguuaaaggaaggcuuuuagaacuuggguucaaggaagauggagaugcgucggaagcucuuuggcgggggugaggaaguucagaaagugugcauuuuccuucuggcauuuaggucuuguccgugugauuugguggugcuugggucauaagccugauuaaaauucagggacauguaccacggcggccaaagcggaauuaauuuuuuuauauggggacuggagcgcugaaaaguuguuccugaccaggcucuaaugagaaauuccucucuccccagguuaugaagacaguauggaguuuccagaccauaguagacauuugcuacagugucugagcgagcagagacaccaggguuuucuuugugacugcacuguucuggugggagaugcccaguuccgagcgcaccgagcuguacuggcuucaugcagcauguauuuccaccucuuuuacaaggaccagcuggacaaaagagacauuguucaucugaacagcgacauuguuacagcccccgcuuucgcucuccugcuugaauucauguaugaagggaaacuccaguucaaagacuugcccauugaagacgugcuagcagcugccaguuaucuccacauguaugacauugucaaagucugcaaaaagaagcugaaagagaaagccaccacggaggcagacagcaccaaaaaggaagaagaugcuucaaguuguucggacaaagucgagagucucuccgauggcagcagccacauagcaggcgauuugcccagugaugaagaugaaggagaagaugaaaaauugaacauccugcccagcaaaagggacuuggcggccgagccugggaacauguggaugcgauugcccucagacucagcaggcaucccccaggcuggcggagaggcagagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcuuccugugaugagagugauguuggcacuaaugacuaugacauggaacauagcacugugaaagaaagugugagcacuaauaacaggguacaguaugagccggcccaucuggcuccccugagggaggacucggucuugagggagcuggaccgggaggacaaagccagugaugaugagaugaugaccccagagagcgagcguguccagguggagggaggcauggagagcagucugcuccccuacgucuccaacauccugagccccgcgggccagaucuucaugugcccccugugcaacaaggucuuccccagcccccacauccugcagauccaccugagcacgcacuuccgcgagcaggacggcauccgcagcaagcccgccgccgaugucaacgugcccacgugcucgcugugugggaagacuuucucuugcauguacacccucaagcgccacgagaggacucacucgggggagaagcccuacacaugcacccagugcggcaagagcuuccaguacucgcacaaccugagccgccaugccguggugcacacccgcgagaagccgcacgccugcaaguggugcgagcgcagguucacgcaguccggggaccuguacagacacauucgcaaguuccacugugaguuggugaacuccuugucggucaaaagcgaagcacugagcuugccuacugucagagacuggaccuuagaagauagcucucaagaacuuuggaaauaauuuuauauauauauaaauaauauauauauauauacauauauauaaauagaucucuauauaguugugguacggucuaaaagcagucuuguuuccuggaaauaaaaaguugggauauuaacuuguuuuugcacuuuagaauagcaugagaaucucacuaauuuagcauucugauaaaagaaacuuuagagcaagucagaauagagagguguuuuuccuuugaggggauaggggaaguaagccaauaagaaccuuuuaaacaaaucguccugucacaaaaugcuuucauauggcuuaauuuugucaacacugcauugucuuuugagcucuuuuuucccccccaacaaaguuuuuuuguuuuuuguuuuuuuuuuuaaguagaaauucccuccaguuuuauuagccucuuuauaugucucaaauugcaugaauuuuuucuggcuguuggaaaccugaaugcuuuuagacccaaauggaaaauuucugaaaugcuggauuaucuauuuuuaaacaagcaguugacuuaaaacuuucuguggcaacuucugguuuucugacaguucccagugagagaaaugcugaaaguacacugggaucacugggacacugucuuaugaagguuugcuugggaugaaaaaggauauugcagcuucagcaguguugaacuguguguuuaaaaaugugaauuacuguuauuguauacuguaauugauuacaugggcugggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuuggaaaaaaaaacugguguuaugaagaacguaaaugcacuguuuuuauuuuuauuuuauauaauuuaaauugacuuucccacugucuuuaaguugaaacuguuaagcugaauaaaaacuuaagcugcaaauugauaacuucgcuacauaacaaggaaaauauaaauguuuacaaacagcuuaaagauuugcaugugcagugugcauuuauaacaaacuucuaauugcacaaaacccaugccagcucagaguuuagguguacacauuuacccaguugagcguucuuagaauaacuacugcacaaguugacaauaggucguucucucuuuuuuuuuguuugcucccuuuuucuuuuucuccccuuccuccuuacccucccucccuuacucuccccccccaccaccacccuccacccccaacucaugaaaagauucuauggacugaaaaagccccaggcugaaaggacuggacugccuugauugacauggggaaggggguuaguagacuauguggauugcggcagcagaggcugcagccuaacgugugguuuuaaugaccagcacgcaaggcaaaagcauuuugcacaguguuuguuuuccugucuugcacuuacaaauaaggucuaugggaguagcauggaaaacguuugcuguuuuucccuuuuuuuuuuaauugcuuuuguuuaaaauuugaucgccuuaacuacuguaaacauagccuauuuuugugcuuaagauacugaauggaaaacuccauuguguguugcuggacuguuuuggaaauauuugguuaaauguguguuaauuuggcuguaauggcauuuaaagcaaacaaacaaacaaaaaaagcugugaaaauggccuuggagcauuaucuuuaguuacuugaagaguuucuaguuuuuuuaaaauacaguuuauguuaaaauaauuuuuauuaauuuagagaagacaaucaaugucugugagaaaacggacuuucuuuuggauuuucuuuuuguggucauugugagugauugcuuuuuccuuuucuuaguuucacauucuuccuuuguucuaaaacuuagacugacaucuagcuuugacaaucauaguauguuuuauuuuccugagggggaauaacuuauaaugcuguuuaguuuuguacuauugguguguuggugaauuuuuaaacugugugcuaacugcaauaaauuauaugaacugagaaaaaaaaaaaaaaaaaaaaaaaaaaaaaaaaaaaaaaaaaaaaaaaaa

SEQ ID NO: 16 - DNA, 1596 bp, Homo sapiens
  CDS (1)..(1596)
atgtgtcctaaaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataa

SEQ ID NO: 17 - protein, 531 aa, Homo sapiens
MCPKGYEDSMEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 18 - DNA, 1569 bp, Homo sapiens
  CDS (1)..(1569)
atggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataa

SEQ ID NO: 19 - protein, 522 aa, Homo sapiens
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 20 - RNA, 3937 nt, Mus musculus
  CDS (260)..(1828)
aggcugcggccgccgcugggcugcugccauggggagccguugaaagaguggggcccuguggcucggccgcgcgccucaggcugccagcgcgggcggggggcggcgggggcgcgcggggcccgggccggggccggcgggcgggcgggggccggcggaggacgcccacaaguccagcggcggcggcggcggcagaggaggaggaggaggaggacgcggcggggacgcggcgacucgggccgcuccguguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaaaaaaaaaaaaaaaaaa

SEQ ID NO: 21 - RNA, 4147 nt, Mus musculus
  CDS (486)..(2054)
aggcugcggccgccgcugggcugcugccauggggagccguugaaagaguggggcccuguggcucggccgcgcgccucaggcugccagcgcgggcggggggcggcgggggcgcgcggggcccgggccggggccggcgggcgggcgggggccggcggaggacgcccacaaguccagcggcggcggcggcggcagaggaggaggaggaggaggacgcggcggggacgcggcgacucgggccgcuccguaaccuccgaggguccgcgguagcgguggacgggucgauccguggaguggaugucuucgggccauugcggugcuugggacucauuaaacugucacucacccccaccaccaccccacugguauuucagaagugcaguuugaguuuguugaugcugaaaguggaaauuugcacuaacgguggcagugugaacuauuuuccugagagguagauccagauaaauugcuuagguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaa

SEQ ID NO: 22 - RNA, 4039 nt, Mus musculus
  CDS (378)..(1946)
aggcugcggccgccgcugggcugcugccauggggagccguugaaagaguggggcccuguggcucggccgcgcgccucaggcugccagcgcgggcggggggcggcgggggcgcgcggggcccgggccggggccggcgggcgggcgggggccggcggaggacgcccacaaguccagcggcggcggcggcggcagaggaggaggaggaggaggacgcggcggggacgcggcgacucgggccgcuccguaaccuccgaggguccgcgguagcgguggacgggucgauccguggaguggaugucuucgggccauugcggugcuugggacucauuaaacugucacucacccccaccaccaccccacuggguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaa

SEQ ID NO: 23 - RNA, 3810 nt, Mus musculus
  CDS (149)..(1717)
ucccgccgggcggaguuaguuaguguggcggagggcaggcggugcgggcucgccucgccucgccuggccgcgcuccccggccggugcagcugcggcgcggggcggacucggugcagcugcggcggcgccucccgguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaa

SEQ ID NO: 24 - RNA, 4312 nt, Mus musculus
  CDS (624)..(2219)
guugcgguugccgggaggccccgggggcuggcgcgcggcggcggcgacgggggagcggucgccgcgccgcuucuugcgacuuuucuuuuguuguugccgcgaccggggucuguuuucucggccguuguggaaggugcuuuccugcugcaccggcucucugcccugcaccuucuuccccgugguuucacccuuccucuuucccugaagugcuuuaugcaccgcgaacugcgggaagauuugcaucuccucucccaccccugaaccucacacacacacccggggcacccugcccccaccucugaaaaacaaacccccuaaccucacagaaccuccgaggguccgcgguagcgguggacgggucgauccguggaguggaugucuucgggccauugcggugcuugggacucauuaaacugucacucacccccaccaccaccccacugguauuucagaagugcaguuugaguuuguugaugcugaaaguggaaauuugcacuaacgguggcagugugaacuauuuuccugagagguagauccagauaaauugcuuagucugcuuuuuuuccaccgauguaacagaccugcagccagcaagacuccgagggaaggacuuaaucaaguuuauguguccuaaagguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaa

SEQ ID NO: 25 - RNA, 4204 nt, Mus musculus
  CDS (516)..(2111)
guugcgguugccgggaggccccgggggcuggcgcgcggcggcggcgacgggggagcggucgccgcgccgcuucuugcgacuuuucuuuuguuguugccgcgaccggggucuguuuucucggccguuguggaaggugcuuuccugcugcaccggcucucugcccugcaccuucuuccccgugguuucacccuuccucuuucccugaagugcuuuaugcaccgcgaacugcgggaagauuugcaucuccucucccaccccugaaccucacacacacacccggggcacccugcccccaccucugaaaaacaaacccccuaaccucacagaaccuccgaggguccgcgguagcgguggacgggucgauccguggaguggaugucuucgggccauugcggugcuugggacucauuaaacugucacucacccccaccaccaccccacuggucugcuuuuuuuccaccgauguaacagaccugcagccagcaagacuccgagggaaggacuuaaucaaguuuauguguccuaaagguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaa

SEQ ID NO: 26 - RNA, 3848 nt, Mus musculus
  CDS (150)..(1745)
agugugugcggaucugugguggagaagguaucucauuccucuccaacaucaucuccacuuugcaucugucucucuuagucugcuuuuuuuccaccgauguaacagaccugcagccagcaagacuccgagggaaggacuuaaucaaguuuauguguccuaaagguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaaaaaaaaaaaa

SEQ ID NO: 27 - RNA, 4954 nt, Mus musculus
  CDS (1283)..(2851)
ucagaagguaucgggguuaggaugaaaguuucacugugucucagcauaguguggaggagagaaguaguuucugaauaguagcuucauauccauccaagaugcacuugcagagguagauguuggcgagcucugccuuuggucccaggauggggaagugggcuccgcugaucuuaagagaguuuuagccacuacagaugaauuuugacugaucuuugccuucccuucuuucacccucucccuugccaaaaaacaugggaaagaugcauaauuggaagaauagaagguacugggaaaacaaaauagaucagguuauuauugaaacaaaguauuuucuguuggauaccugaggcagagccagaaaaaaaaguuuuauuuauuuuauuuuuucuaggaaggcuuuaaaaaauauuuaacuuacaaaguuuuugcaccucauuuuacagugguaaaaguuugauuaauuuuaauaugaaguuccuuuugcccuauagcagaucaaaguucagaugaaaauguucagaguuaacagacucucugguugcuaauguuguugagguagaggaucuacaaagaaacaggaccccccccccauucauuuuuaaaauguuuggauuuguucauaguaaagaugaucuuucccaucugucgguacaguucugcuccucuguguguguaguaccugcagaucuuaccgcaccaacaucuguucuuauacacagggcuuuuguuguuguaacacucugacuuuaaaaguaaguuguuugcugauuucugcacuaauuaugcaauuucauuuuuuuuccccuucauuucagccaaauguguguguucagucugugcuuucuaaguaguuggguaucagacguguuacaaagaauggcuggacaugucuucuugacucuuccagaauuugugugggacuuugccugauauuaugaguucuuucccuuugaaguuaaagcucucuuuuagguuuuguaggaugucauuugaugaguuguugguuaaauuucuggaagaggucugaguucuuguucuaagucauaagcuaaaggaagcuuuaaacuugaccaggaguuaugaguggggugagcaaagccuuggugauugcauuuagguugaauguguggcuugggaguccuuggggugugagccugauuuaaaauucagacauguaacucuccaccacuaaggcggauauuuuuuaaaguggcgacuagagucuaaauuccugacuaggcucuaacgagaaacuccucuuuccccagguuaugaagacaguauggaguuuccagaccacagcagacauuugcugcagugucugagcgagcagaggcaccagggcuuucuuugugacugcacuguucuggugggagaugcccaguuccgggcacaccgagcuguucuggcuucaugcagcauguauuuccaccucuuuuacaaggaccagcuggacaaaagagacauuguucaucugaacagcgacauugugacagcccccgcuuucgcucuccugcuugaauucauguacgaagggaaacuccaguucaaagacuugcccauugaggacgugcuagcagcugccaguuaucuccacauguaugacauugucaaagucugcaaaaagaagcugaaagagaaagcuaccacagaggcggacagcaccaaaaaagaagaagaugcuucaaguuguucggauaaagucgagagccucucagacggcagcagccacauggcaggugaccugcccagugaugaagaugaaggcgaagaugacaaauugaacauucuacccagcaaaagggacuuggcagcugagccugggaacauguggaugcgauugcccucagacucagcaggcaucccccaggcuggcggagaggccgagccacacgccacagcagcuggaaaaacaguagccagccccugcagcucaacagagucuuugucccagaggucugucaccuccgugagggauucggcagauguugacugugugcuggaccugucugucaaguccagccuuucaggaguugaaaaucugaacagcucuuauuucucuucacaggacgugcugagaagcaaccuggugcaggugaagguggagaaagaggcguccugugaugagagugauguuggcacuaaugacuaugacauggaacauagcacugugaaagagagugugagcacuaacaacaggguacaguaugagccagcccaccuggcuccucugagggaggacucggucuuaagggaacuggaucgggaggacaaagccagcgaugaugagaugaugaccccagagagcgagcggguccagguggaagggggcauggagaacagucugcuucccuacgucuccaauauccugagccccgcaggccagaucuucaugugcccccugugcaacaaagucuuccccagcccccacauccugcagauccaccugagcacgcacuuccgagagcaggauggcauccguagcaagccugccgccgaugucaaugugcccacgugcucccugugugggaagacuuucuccugcauguacacccucaagcgccacgagaggacucacucgggggagaagcccuacacgugcacccaaugcggcaagaguuuccaguacucgcacaaccugagccgucaugccguggugcacacccgcgagaagccccaugccugcaaguggugcgagcgcagguucacgcaguccggagaccuguacagacacauccgcaaguuccacugugaguuggugaacuccuugucggucaaaagugaagcgcugagcuugcccacugucagagacuggaccuuagaagauagcucucaagaacuuuggaaauaauuuuauauauauauauauauaaauaauauauauauauauauauacauauauauacauagaucucuauauaguugugguacggucuaaaagcagucuuguuuccuggaacugaaaaguugggaucuuaacuuguuuuugcacuuuagaauaauagcaugagaaucucacuaauuuagcauucugauaaaagaaacuagagcaagucacaaguagagagcugucuuucuuuugaggguuaggccaaguuagccaauaaaaccuucucaacaaaugguucugucacaaacgcuuucacauggcuuacuuuuguacacacugcauugucuugagcgcuuucccugugcuccacacuguuuuguuuuguuuuguuuucagaguagaaauccccuccaguuuauuagccucuuuauaugucucaaauugcaugaauauuuucuggcuguuggaaaccugaaugcuuuuagacccaaauggaaaauuucugaaaugcuggauuaucuauuuuuaaacaagcaguugacuuaaaacuuuguguggcaacuucugguuuucugaugguccccagugagagaaaucugacaguacacugggucacugggacugucucugugaagguuugcuugagaugaaaaaggauauugcagcuucagcaguguugaacuguguguuuaaaaugugaauuacuguucuuguauacuguaauugauuacacgggcuggggggggugucaaagaacuugacagguuguguugaugcucuuaguugagucuugaaaaguaaauauuaacgcuacagaaaugcaugaguuucaauauauuuuuugucuuuguuugcauuguauaacuuuaacgagugaguuuaaaauuauuuaauuuccuuagaaaaauagcaccauuugggaaaaaaacugguguuacgaagaacauaaaugcacugucuuuauuuuuauuuuuauuuuauauaauuuaaauugacuuuucccacgucuuuaaguuugaaacuguuaagcugaauaaaaacuuaagcugcaaauugauaacuucgcuacauuacaaggaacauauaaauguuuacaaacggcuuaaagaauugcaugugcagugugcauucauaccacacuuguaauugcacagaacccaugccagcucagaguuuagguguacacacguacccagccgaguguuuuuagaauaacuacugcacaaauugacaauaggucauaggucauucucucucucccuccuuuuugcuuccccuuucuuuuucuccccuauuuccuuuccuucccccuacccccuaacucaugaaaagauucuauggacugaaaaagccccaggcugaaaggacugaaccgccuugauugacauggggagggggguuaguagacuguguaucggcagcagaggcugcagcccgaugugugguucugauggccagcgcggggcagaagcguuuugcacacuguuuguuuuccugucuugcacuuacaaauaaggucuaugggaguagcaugggaaacagugguuguuuuucccuuuuucuuuuuuaauugcuuuuguuuaaaauuugaucgccuuaacuacuguaaacauagccuauuuuugugcuuaagauacugaauggaaaacuccauuguguguugcuggacuguuuuggaaauauuuggucaaauguauguuaauuuggcuguaauggcauuuaaagcaaacaaacaaacaaacaaacaaagcugugaaaauggccuuggagcauuaucuuuaguuacuugaagaguuucuaguuuuuuuuuuuuuaauacaguuuauguuaaaauaaucuuuauuaaucuagaggagacaaucaaugucuguggaagaacggacuuuuuuggauuuucuuuuuuaauggucauugugagugauugcucuuucuuuuacuuaguuucacauucuuccuuuguucuaaaucuuagacugacaucuagcuuugacaaucauaguauguuuuauuuccugagggggaauaacuuauaaugcuguuuaguuuuguacuauugguguguuggugaauuuuuaaacugugugcuaacugcaauaaauuauaugaacugagaaaaaaaaaaaa

SEQ ID NO: 28 - DNA, 1596 bp, Mus musculus
  CDS (1)..(1596)
atgtgtcctaaaggttatgaagacagtatggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataa

SEQ ID NO: 29 - protein, 531 aa, Mus musculus
MCPKGYEDSMEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 30 - DNA, 1569 bp, Mus musculus
  CDS (1)..(1569)
atggagtttccagaccacagcagacatttgctgcagtgtctgagcgagcagaggcaccagggctttctttgtgactgcactgttctggtgggagatgcccagttccgggcacaccgagctgttctggcttcatgcagcatgtatttccacctcttttacaaggaccagctggacaaaagagacattgttcatctgaacagcgacattgtgacagcccccgctttcgctctcctgcttgaattcatgtacgaagggaaactccagttcaaagacttgcccattgaggacgtgctagcagctgccagttatctccacatgtatgacattgtcaaagtctgcaaaaagaagctgaaagagaaagctaccacagaggcggacagcaccaaaaaagaagaagatgcttcaagttgttcggataaagtcgagagcctctcagacggcagcagccacatggcaggtgacctgcccagtgatgaagatgaaggcgaagatgacaaattgaacattctacccagcaaaagggacttggcagctgagcctgggaacatgtggatgcgattgccctcagactcagcaggcatcccccaggctggcggagaggccgagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcgtcctgtgatgagagtgatgttggcactaatgactatgacatggaacatagcactgtgaaagagagtgtgagcactaacaacagggtacagtatgagccagcccacctggctcctctgagggaggactcggtcttaagggaactggatcgggaggacaaagccagcgatgatgagatgatgaccccagagagcgagcgggtccaggtggaagggggcatggagaacagtctgcttccctacgtctccaatatcctgagccccgcaggccagatcttcatgtgccccctgtgcaacaaagtcttccccagcccccacatcctgcagatccacctgagcacgcacttccgagagcaggatggcatccgtagcaagcctgccgccgatgtcaatgtgcccacgtgctccctgtgtgggaagactttctcctgcatgtacaccctcaagcgccacgagaggactcactcgggggagaagccctacacgtgcacccaatgcggcaagagtttccagtactcgcacaacctgagccgtcatgccgtggtgcacacccgcgagaagccccatgcctgcaagtggtgcgagcgcaggttcacgcagtccggagacctgtacagacacatccgcaagttccactgtgagttggtgaactccttgtcggtcaaaagtgaagcgctgagcttgcccactgtcagagactggaccttagaagatagctctcaagaactttggaaataa

SEQ ID NO: 31 - protein, 522 aa, Mus musculus
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHMAGDLPSDEDEGEDDKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMENSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 32 - DNA, 3428 bp, Artificial
  Recombinant DNA
ataacttcgtatagcatacattatacgaagttatctgaagttcctatactttctagagaataggaacttctcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatctggacgtaaactcctcttcagacctgaagttcctatactttctagagaataggaacttcggaatttgatcctcgagatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgaagttcctatactttctagagaataggaacttctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctataacttcgtatagcatacattatacgaagttat

SEQ ID NO: 33 - DNA, 3489 bp, Artificial
  Recombinant DNA
gtcgacaagcttcccagcggccgcgtggatccacctaataacttcgtatagcatacattatacgaagttatctgaagttcctatactttctagagaataggaacttctcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatctggacgtaaactcctcttcagacctgaagttcctatactttctagagaataggaacttcggaatttgatcctcgagatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgaagttcctatactttctagagaataggaacttctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctataacttcgtatagcatacattatacgaagttattgataaatacgtgctagtgccacc

SEQ ID NO: 34 - DNA, 3467 bp, Artificial
  Recombinant DNA
gcggccgcgtggatccacctaataacttcgtatagcatacattatacgaagttatctgaagttcctatactttctagagaataggaacttctcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatctggacgtaaactcctcttcagacctgaagttcctatactttctagagaataggaacttcggaatttgatcctcgagatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgaagttcctatactttctagagaataggaacttctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctataacttcgtatagcatacattatacgaagttattgataaatacgtgctagt

SEQ ID NO: 35 - DNA, 118 bp, Mus musculus
aacctccgagggtccgcggtagcggtggacgggtcgatccgtggagtggatgtcttcgggccattgcggtgcttgggactcattaaactgtcactcacccccaccaccaccccactgg

SEQ ID NO: 36 - DNA, 84 bp, Mus musculus
tctgctttttttccaccgatgtaacagacctgcagccagcaagactccgagggaaggacttaatcaagtttatgtgtcctaaag